(19) United States
(12) Patent Application Publication
(10) Pub. No.: US 2015/0010525 A1
(43) Pub. Date: Jan. 8, 2015

(54) Catalytic Tagging System to Study Macro-Molecular Interactions Using Engineered Ubiquitin Ligase and Ubiquitin-Like Proteins to Facilitate Substrate Identification

(71) Applicant: THE REGENTS OF THE UNIVERSITY OF CALIFORNIA, Oakland, CA (US)

(72) Inventors: James A. Wells, Burlingame, CA (US); Min Zhuang, San Francisco, CA (US)

(21) Appl. No.: 14/371,533

(22) PCT Filed: Jan. 23, 2013
(86) PCT No.: PCT/US2013/022800

§ 371 Date: Jul. 10, 2014

Related U.S. Application Data
(60) Provisional application No. 61/589,797, filed on Jan. 23, 2012.

(51) Int. Cl. G01N 33/68; G01N 33/58; C12N 9/16; G01N 33/94; C12N 9/00

(52) U.S. Cl. CPC G01N 33/6848; G01N 33/94; C12N 9/93; C12N 9/16; G01N 33/58; C07K 2319/00; C12Y 603/02019; C07K 2319/95; C12Y 301/03016; G01N 2500/10; G01N 2500/02; G01N 2458/15
USPC 424/94.6; 435/7.6; 435/183; 506/9

(57) ABSTRACT

The present invention describes an unbiased catalytic tagging system to search for target substrates. Identification of substrates for specific baits is accomplished by utilizing an orthogonal system consisting of an E1 activating enzyme, an E2 ubiquitin-like conjugating enzyme, and baits that are fused to the E2 ubiquitin-like conjugating enzyme. The present invention thus reveals important mutually antagonistic substrates of specific baits.

### CROSS-REFERENCE TO RELATED APPLICATIONS

This application claims priority to U.S. Provisional Application No. 61/589,797 filed on Jan. 23, 2012, the disclosure of which are incorporated herein by reference in their entirety for all purposes.

### STATEMENT AS TO RIGHTS TO INVENTIONS MADE UNDER FEDERALLY SPONSORED RESEARCH OR DEVELOPMENT

This invention was made with government support under grant no. R01 GM097316 awarded by the National Institutes of Health. The government has certain rights in the invention.

### BACKGROUND OF THE INVENTION

Macro-molecular interactions, including protein-protein, protein-lipid, protein-nucleic acid and protein-small ligand interactions, are critical for the majority of biological functions for almost every living system. However, macro-molecular interactions are non-covalent, and are usually reversible and transient. These properties make such interactions difficult to detect using traditional methods.

Ubiquitination is a post-translational modification that plays an important role in protein level control as well as signal transduction. Ubiquitin is attached to target proteins by a three-step mechanism involving the sequential actions of E1, E2 and E3 enzymes. The ubiquitin cascade is pyramidal in design. In humans, two E1-activating enzymes transfer ubiquitin to roughly three dozen E2 enzymes, which function together with several hundred different E3 ubiquitin ligases to ubiquitinate thousands of substrates. Determining the direct substrates of individual ubiquitin ligases is important in understanding how ubiquitination exerts its effect on cellular functions. Genetic studies have revealed more than 600 E3 genes, specified by a RING or HECT domain, while proteomic studies have identified thousands of ubiquitin-modified proteins. However, the linkage between individual E3 and their specific substrates is largely missing. It is challenging to identify the substrates of a specific E3, largely because of the weak interaction between E3 and substrates, the heterogeneity of modifications, and the rapid degradation of ubiquitinated substrates. Most ubiquitin substrate identification approaches involve hypothesis-driven candidate approaches, which are slow and heavily biased.

To address the challenges in study ligase-substrates interactions, a novel approach is described herein using a catalytic tagging system that combines protein engineering and SILAC mass spectrometry to screen substrates of specific ubiquitin ligases or other bioactive molecules including small molecule compounds. Ubiquitin ligases are engineered to allow transfer of an ubiquitin-like protein instead of ubiquitin. The substrates of this particular ligase can then be marked with the ubiquitin-like protein and can be enriched and identified by mass spectrometry.

This novel approach converts the non-covenant protein-protein interaction into a covalently linked ubiquitin-like protein tag. This approach facilitates the identification of substrates of E3 ligases and presents a strategy for the generation of similar methods to screen substrates for other ubiquitin-like protein enzymes. It can further be generalized and applied to other macro-molecular interactions.

### BRIEF SUMMARY OF THE INVENTION

In an embodiment, the present invention provides an assay for the identification of a substrate. The assay comprises fusing a bait to an E2 ubiquitin-like conjugating enzyme wherein under wild-type conditions said E2 ubiquitin-like conjugating enzyme conjugates a ubiquitin-like protein other than ubiquitin; expressing in a cell said bait fused to said E2 ubiquitin-like conjugating enzyme wherein the cell comprises one or more ubiquitin-like proteins; and identifying a substrate, wherein said substrate binds ubiquitin under wild-type conditions and is identified by selecting said substrate bound to said one or more ubiquitin-like proteins. In an embodiment, the bait comprises a peptide, a protein, a small molecule, a nucleic acid, or a carbohydrate. In an embodiment, the bait is a modified version of an E3 ligase and wherein the E3 ligase lacks a RING domain. In an embodiment, the E3 ligase is selected from the group consisting of XIAP, cIAP1, CRBN, gp78, Doa10, RNF13, RNF38, TEB4, RNF139, RNFx2, RNF126, Hrd1, and MARCH1. In an embodiment, the E3 ligase is XIAP. In an embodiment, the assay of claim 4, wherein the E3 ligase is cIAP1. In an embodiment, the modified version of the E3 ligase comprises a substrate binding domain and wherein the modified version of the E3 ligase is capable of fusing to an E2 ubiquitin-like conjugating enzyme. In an embodiment, the bait is a phosphatase. In an embodiment, the bait is a kinase. In an embodiment, the bait is a small molecule. In an embodiment, the small molecule is selected from the group consisting of dasatinib, imatinib, nilotinib, thalidomide, lenalidomide, and pomalidomide. In an embodiment, the small molecule is dasatinib. In an embodiment, the ubiquitin-like protein is selected from the group consisting of NEDD8, SUMO, ISG15, ATG8, ATG12, FAT10 and functional equivalents thereof. In an embodiment, the ubiquitin-like protein is NEDD8. In an embodiment, the E2 ubiquitin-like conjugating enzyme is selected from the group consisting of NEDD8 E2, SUMO E2, ISG15 E2, ATG8 E2, ATG12 E2, FAT10 E2, and functional equivalents thereof. In an embodiment, the E2 ubiquitin-like conjugating enzyme is NEDD8 E2 or a functional equivalent thereof. In an embodiment, the NEDD8 E2 is Ubc12. In an embodiment, the substrate is specific for the bait. In an embodiment, the identifying comprises taking a cell extract and screening candidate substrates using mass spectrometry. In an embodiment, the mass spectrometry is SILAC mass spectrometry. In an embodiment, the mass spectrometry is liquid chromatography-tandem mass spectrometry (LC/MS/MS).

In another embodiment, a composition is provided. The composition comprises a bait, wherein said bait is fused to an E2 ubiquitin-like conjugating enzyme capable of conjugating a ubiquitin-like protein other than ubiquitin. In an embodiment, the bait comprises a peptide, a protein, a small molecule, a nucleic acid, or a carbohydrate. In an embodiment, the bait is a modified version of an E3 ligase and wherein the E3 ligase lacks a RING domain. In an embodiment, the E3 ligase is selected from the group consisting of XIAP, cIAP1, gp78, Doa10, RNF13, RNF38, TEB4, RNF139, RNFx2, RNF126, Hrd1, and MARCH1. In an embodiment, the E3 ligase is XIAP. In an embodiment, the E3 ligase is cIAP1. In an embodiment, the modified version of the E3 ligase comprises a substrate binding domain and wherein the modified version of the E3 ligase is capable of fusing to an E2 ubiquitin-like conjugating enzyme. In an embodiment, the bait is a phosphatase. In an embodiment, the bait is a kinase. In an embodiment, the bait is a small molecule. In an embodiment, the small molecule is selected from the group consisting of dasatinib, imatinib, nilotinib, thalidomide, lenalidomide, and pomalidomide. In an embodiment, the small molecule is dasatinib. In an embodiment, the E2 ubiquitin-like conjugating enzyme is selected from the group consisting of NEDD8 E2, SUMO E2, ISG15 E2, ATG8 E2, ATG12 E2, FAT10 E2 and functional equivalents thereof. In an embodiment, the E2 ubiquitin-like conjugating enzyme is a NEDD8 E2 or a functional equivalent thereof. In an embodiment, the NEDD8 E2 is Ubc12. In an embodiment, the ubiquitin-like protein is selected from the group consisting of NEDD8, SUMO, ISG15, ATG8, ATG12, FAT10, and functional equivalents thereof. In an embodiment, the ubiquitin-like protein is NEDD8 or a functional equivalent thereof. In an embodiment, the NEDD8 is biotinylated.

In an embodiment, a kit is provided. The kit comprises a bait, wherein said bait is fused to an E2 ubiquitin-like conjugating enzyme capable of conjugating a ubiquitin-like protein other than ubiquitin. In an embodiment, the kit further comprises a ubiquitin-like protein selected from the group consisting of NEDD8, SUMO, ISG15, ATG8, ATG12, FAT10, and a functional equivalent thereof. In an embodiment, the ubiquitin-like protein is NEDD8 or a functional equivalent thereof.

In an embodiment, a method of inducing apoptosis is provided. The method comprises administering to a subject an effective amount of one or more substrates identified by the assay described herein wherein the bait is an E3 ligase. In an embodiment, the substrate is PGAM5.

### BRIEF DESCRIPTION OF THE DRAWINGS

FIG. 1 illustrates the role of IAPs in apoptosis.

FIG. 2 illustrates the enzymatic cascade for Ubiquitination.

FIG. 3 illustrates engineering of ubiquitin ligase to transfer UBL.

FIG. 4 illustrates the advantages of the present invention.

FIG. 5 illustrates the design and validation of an exemplary catalytic tagging system as embodied by the present invention. (A) Schematic view of wild type RING ubiquitin ligase. A typical RING ubiquitin ligase (depicted as RING and SBD) contains a RING domain and a substrate-binding domain (SBD). The RING domain binds a ubiquitin E2 (Ub E2) and facilitates transfer of ubiquitin (Ub) from the catalytic cysteine on the E2 to lysines on substrate protein (S) recruited by SBD. (B) Schematic view of an exemplary catalytic tagging system referred to as the NEDDylator. The RING domain of an ubiquitin ligase is removed and NEDD8 E2 Ubc12 is fused to the substrate binding domains (SBD) of the ligase to generate the NEDDylator (depicted as N8 E2 and SBD). This brings the NEDD E2 conjugated with NEDD8 (N8) in close proximity to the substrate of the original E3 ligase. (C) Wild type XIAP has ubiquitin specific ligase activity for self and substrate ubiquitination. Immunoblots against XIAP and caspase-7 detect ubiquitinated proteins. (D) The exemplary NEDDylator^{XIAP} specifically mediates transfer of NEDD8 and not ubiquitin. The exemplary NEDDylator^{XIAP}, instead of XIAP, was added in the same assay as in (C).

FIG. 6 illustrates how the activity of an exemplary catalytic tagging system is dependent on close proximity between the XIAP substrate binding domain and Ubc12, but not restricted by the relative orientation between the XIAP substrate binding domain and Ubc12. (A) Schematic view of exemplary catalytic tagging systems NEDDylator^{XIAP} and NEDDylator^{XIAP}-R. (B) In vitro NEDDylation of caspase-7 by NEDDylator^{XIAP} and NEDDylator^{XIAP}-R produce similar results. Caspase-7 was NEDDylated in the presence of NEDD8 E1 (NAE1) and the NEDDylator fusions. The anti-caspase-7 antibody only recognizes the large subunit Casp-7(L). Casp-7(L)˜N8 represents the NEDD8 modified form of the large subunit of caspase-7. (C) Schematic view of the split NEDDylator, where Ubc12 is fused to FRB, and XIAP is fused to FKBP. (D) XIAP is NEDDylated in the presence of 1 μM rapamycin with 18 combinations of split NEDDylators. Immuno-blotting was performed with an anti-XIAP antibody. (E) Caspase-7 is NEDDylated in the presence of 1 μM rapamycin with 18 combinations of split NEDDylators. Immuno-blotting was performed with an anti-caspase-7 antibody. In (D) and (E), DMSO was added to control samples.

FIG. 7 illustrates validation of an exemplary catalytic tagging system. (A) NEDDylator^{cIAP1} specifically modifies active caspase-7 in vitro with NEDD8 but not with ubiquitin. (B) The NEDDylator functions in cell lysates. Apoptotic cell lysates were generated by treatment of Jurkat cells with 1 μM STS for 4 hours. Recombinant NEDDylators, together with HB-NEDD8 and ATP, were added at 37° C. for 1 hour (right panel). The HB-NEDD8 modification of caspase-7 was examined by immuno-blotting. The NEDDylator^{FRB}, where FRB was fused to Ubc12 instead of XIAP¹⁻⁴³⁴, was used as a negative control. The same experiment was done with regular cell lysate where majority of caspase-7 was in the un-cleaved procaspase-7 form (left panel). (C) NEDDylator^{XIAP} and HB-NEDD8 were co-transfected in Jurkat cells and cultured in the presence of 4 μM biotin. 24 hours after transfection, cells were first treated with 1 μM STS for 4 hours, then lysed and immuno-precipitated with either anti-caspase-7 (Cell Signaling, 9491) or anti-SMAC (Cell Signaling, 2954) antibody. The modification of both proteins was examined by StrepAvidin-HRP based detection of the biotin tag. The asterisk indicates the position of the immunoglobulin light chain.

FIG. 8 illustrates the identification of ubiquitination sites on caspase-7. (A) Caspase-7 protein sequence is displayed with peptides identified by LC-MS/MS. In vitro ubiquitinated caspase-7 was trypsinized and subjected to LC-MS/MS for identification of ubiquitination sites by the characteristic Gly-Gly on the ε-amino groups after trypsin digestion. (B) GlyGly modified peptides identified from ubiquitinated caspase-7. NEDDylation generates similar GlyGly modified peptides after trypsin digestion. The highlighted peptides were also identified from caspase-7 that was neddylated by NEDDylator^{XIAP}.

FIG. 9 illustrates the identification of potential XIAP and cIAP1 substrates using respective exemplary catalytic tagging systems and SILAC-based mass spectrometry. (A) Workflow for comparing proteins that are NEDDylated with (“Light”) or without (“Heavy”) the NEDDylator. (B) Distribution of potential XIAP substrate protein Light/Heavy ratios obtained by the SILAC experiments. Each dot represents a protein, from which at least two unique peptides were identified. Light/Heavy ratios of each protein are plotted against the log scale of summed intensity. Known XIAP substrates caspase-7, HtrA2 and Smac are shown. (C) Distribution of potential cIAP1 substrate protein Light/Heavy ratios obtained by a similar SILAC experiment. Labels are the same as in (B).

FIG. 10 illustrates the purification of NEDDylated IAP substrates.

FIG. 11 illustrates the identification of known IAP substrates Smac and caspase-7 from cell lysate using an exemplary catalytic tagging system, and statistical analysis of SILAC data. (A) Silver stained SDS-PAGE gel shows the purification of NEDDylated proteins from apoptotic cell lysates. FT—flow through; SA—StreptAvidin beads. (B) Immuno-blotting with caspase-7 and Smac shows the purification and enrichment of the NEDDylated form with tandem affinity pull downs. 1—lysate treated with Ubc12; 2—lysate treated with the NEDDylator^{XIAP}; 3—lysate treated with the NEDDylator^{cIAP1}. (C) Protein sequences of caspase-7 and Smac are displayed with peptides identified in SILAC experiments highlighted in gray. (D) Histogram of the distribution of 247 cullin peptide L/H ratios identified in XIAP_SILAC experiment (left) and distribution of potential XIAP substrate protein L/H ratios obtained by the SILAC experiments (right). L/H ratio mean and standard deviation (SD) are calculated from cullin peptides. On the right, L/H ratios of each protein are plotted against the summed intensity in log scale with known XIAP substrates. The vertical dashed lines indicate the range of one SD. (E) Histogram of the distribution of 231 cullin peptide L/H ratios identified in cIAP1_SILAC experiment (left) and distribution of potential cIAP1 substrate protein L/H ratios obtained by the SILAC experiments (right).

FIG. 12 illustrates the identification of the proteolytic form of PGAM5 as an XIAP and cIAP1 substrate in vitro. (A) PGAM5 protein sequence is displayed with peptides identified by mass spectrometry highlighted in gray. Overall, 15 unique peptides from PGAM5 were recovered and account for 43% of the entire protein sequence. (B) An example of LC-MS/MS spectrum from endogenous PGAM5 N-terminal peptide after subtiligase labeling and positive enrichment. A non-natural amino acid, 2-aminobutyric acid (Abu), is a designed tag showing characteristic labeling by subtiligase. (C) PGAM5 is cleaved into a truncated form in cells. Jurkat cells were transiently transfected with C-terminal Myc tagged full length PGAM5 or PGAM5 (Δ2-24). Immuno-blot with anti-Myc antibody shows an N-terminal cleaved form of PGAM5 with molecular weight identical to PGAM5(Δ2-24). (D) N-terminal amino acid sequence alignment of PGAM5(Δ24) and other known evolutionarily conserved IBM sequences. Reaper, Hid, and Grim are Drosophila proteins that bind DIAP1, the Drosophila IAP. Amino acids are colored according to their chemical properties. (E) XIAP binds PGAM5(Δ24) but not the full length PGAM5. Recombinant GST fused XIAP, PGAM5(FL), and PGAM5(Δ24) were used for in vitro GST pull down experiments. (F) PGAM5(Δ24) binds to XIAP and cIAP1 independent of the RING domain. GST, GST fused XIAP(ΔR), or GST fused cIAP1(ΔR) were first immobilized on glutathione sepharose beads, then mixed with equal amount to PGAM5(Δ24) for a short time incubation. The resins were washed and proteins were eluted and analyzed with SDSPAGE and coomassie blue staining. (G) PGAM5(Δ24) but not the full length protein can be ubiquitinated by both XIAP and cIAP1. PGAM5(FL) and PGAM5(Δ24) containing a C-terminal His tag were expressed and purified from bacteria. The ubiquitination assay was performed in vitro and PGAM5 is detected with an anti-His antibody.

FIG. 13 illustrates functional studies of PGAM5 in cells. (A) PGAM5(Δ24) binds XIAP and cIAP1 in vivo. On the left, HEK293 cells were cotransfected with Flag-cIAP1 and PGAM5(FL)-Myc or PGAM5(Δ24)-Myc. Total cell lysates were analyzed by immune-blotting with anti-Flag and anti-Myc antibodies (bottom two panels). Anti-Myc immune-precipitates (IP) were subjected to immunoblot analysis using anti-Myc and anti-Flag antibodies (top two panels). On the right, PGAM5(Δ24)-Myc was transiently expressed in HEK293 cells. 36 hours after transfection, cells were lysed and subjected for Immuno-precipitation with either anti-Myc antibody or an isotype control antibody (mouse IgG). Proteins in the whole cell lysate (bottom three panels) and proteins that bind to the antibody conjugated sepharose beads (top three panels) were assayed with immunoblotting. An asterisk indicates the position of the immunoglobulin heavy chain. (B) XIAP and cIAP1 stimulate PGAM5(Δ24) ubiquitination in vivo. HeLa cells were transiently co-transfected with HA-Ubiquitin, PGAM5(Δ24)-Myc, with or without Flag-IAP construct. Ubiquitination of PGAM5(Δ24)-Myc were addressed by immuno-precipitating with Myc antibody then blotted with an HA antibody. (C) Cytosolic PGAM5(Δ24) is increased in apoptotic cells. Jurkat and HeLa cells were treated with 1 μM STS for 4 hours. The cytosolic protein extracts were used for immuno-blotting analysis with anti-PGAM5 antibody. (D) Time course of increasing PGAM5(Δ24) in cytosol. Jurkat cells were treated with 1 μM STS and harvested at different time points to extract cytosolic proteins for immunoblotting analysis with anti-PGAM5 antibody. (E) High PGAM5 expression induces apoptosis. Jurkat cells were co-transfected with indicated PGAM5 constructs and GFP at a 5:1 molar ratio. 24 hours after transfection, cell viability was measured by Annexin V staining. Different expression levels of GFP were gated to indicate the expression of PGAM5. Error bars represent SEM calculated from triplicate experiments. Representative anti-Myc immuno-blotting from one of the three experiments shows the relative expression levels of PGAM5(FL) and PGAM5(Δ24). (F) PGAM5 sensitizes cells to the apoptosis inducer STS. HeLa cells are transiently transfected with indicated PGAM5 constructs in 96 well format. 24 hours after transfection, cells are treated for 2 hours with STS at different concentrations. Cell viability is then immediately measured by CellTiter-Glo luminescent assay. Percentages of apoptotic cells are normalized with DMSO treated samples. PGAM5 levels are analyzed with anti-Myc immune-blotting before STS treatment. Error bars represent SEM calculated from triplicate samples.

FIG. 14 illustrates strategies to obtain recombinant PGAM5 (Δ24) and express PGAM5 (Δ24) in mammalian cells, and functional studies of PGAM5(Δ24). (A) Schematic view of PGAM5(FL) and PGAM5(Δ24) constructs used for bacteria expression and purification. (B) Electrospray ionization mass spectrometry analysis of the purified recombinant PGAM5(Δ24) shows that the first Met is removed. (C) Schematic view of PGAM5(Δ2-24) and Ub-PGAM5(Δ24) constructs used for mammalian cell transfection and expression. (D) Ubiquitin is cleaved from PGAM5(Δ24) in cells. Immuno-blotting shows the same sized protein product with either PGAM5(Δ2-24) or Ub-PGAM5(Δ24) transfection of HeLa cells. (E) MG132 inhibition increases cellular PGAM5(Δ24) ubiquitination. HeLa cells are transiently co-transfected with HA-Ubiquitin, PGAM5(Δ24)-Myc, with or without XIAP or the treatment of MG132. Ubiquitination of PGAM5(Δ24)-Myc are addressed by immunoprecipitating with Myc antibody then blotting with HA antibody. (F) GFP plasmid is co-transfected with indicated PGAM5 constructs into Jurkat cells. In the flow cytometry dot plots (the upper panel), cells with different expression levels of PGAM5 are gated out according to GFP level. The histograms (lower panel) show the cell viability measured by Annexin V staining.

FIG. 15 illustrates reverse mediation of NEDDylation of IAPs by the exemplary catalytic tagging system NEDDylator^{PGAM5(Δ24)}. (A) Designs of non-ligase NEDDylators. (B) NEDDylator^{PGAM5(Δ24)} induces NEDD8 modification of XIAP and cIAP1. Recombinant NEDDylator^{PGAM5(Δ24)} (with GGSG linker), XIAP(ΔR) (residues 1-434) and cIAP1(ΔR) (residues 1-555) were expressed and purified to be used in in vitro NEDDylation assays.

FIG. 16 illustrates the similarities of PGAM5 with other IAP substrates.

FIG. 17 illustrates the pro-apoptotic role of PGAM5.

FIG. 18 illustrates proteolytic cleavage that generates an N-terminal IAP binding Motif (IBM) common to IAP substrates. (A) Summary table of potential XIAP and cIAP1 substrates containing IBM-like sequences from combined data sets. Gene names are used to represent each protein. (B) Sequence logo representation of the frequency of amino acids in the IBM-like cleavage sites identified in 16 mitochondrial proteins listed in summary table. Amino acids are colored according to their chemical properties. (C) Sequence alignment of amino acid residues surrounding the caspase cleavage sites. Caspases cleave at Asp and generate IBM-like sequences. (D) A model for IAP-IBM interactions.

FIG. 19 illustrates a catalytic tagging system optimized for small molecule target identification. (A) Representation of a catalytic tagging system optimized for small molecule target identification. (B) A linear diagram of two catalytic tagging system constructs.

FIG. 20 illustrates NEDDylation of ABL-3D in vitro. ABL-3D is a truncated form of ABL, containing only the SH3, SH2, and kinase domains. (a) ABL-3D is NEDDylated by a catalytic tagging using dasatinib as bait (NEDDylator^{Dasatinib}) at different concentrations. NEDDylation was determined by the appearance of higher molecular weight bands when immunoblotting for ABL. (B) Free dasatinib is able to inhibit NEDDylation of ABL in a dose dependent manner, indicating that NEDDylation of ABL by NEDDylator^{Dasatinib} is driven by the affinity to ABL.

FIG. 21 illustrates structures of ligand derivatives for optimization for small molecule target identification.

FIG. 22 illustrates the basic catalytic tagging system concept.

### DETAILED DESCRIPTION OF THE INVENTION

### Introduction

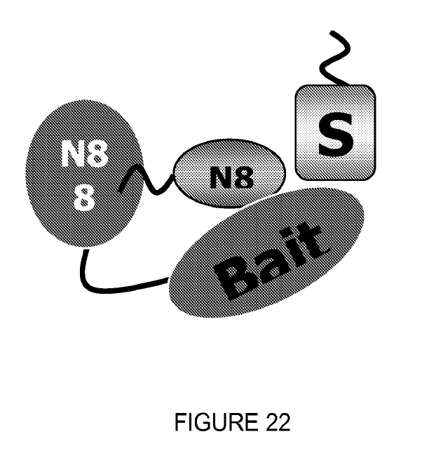

Described herein are catalytic tagging systems. The conceptual embodiment of the catalytic tagging system described herein is a bait that is fused to an ubiquitin-like E2 ligase. The E2 ligase conjugates a ubiquitin-like protein to a binding partner of the bait, which allows subsequent determination of target candidates that interact with the bait (see FIG. 22).

An exemplary catalytic tagging system of the present invention provides an unbiased proteomics approach to search for E3 ligase substrates that are of central importance in regulating cell death and inflammatory signaling. The catalytic tagging systems described herein provide important mutually antagonistic substrates and a powerful approach for hunting substrates for E3 ligases or other protein-protein partners. The catalytic tagging systems described herein further provide an unbiased approach to search for targets of small molecule compounds or any other bioactive molecule.

Identification of substrates for specific E3 ligases is extremely challenging because of the weak transient interaction between E3s and their substrates, the tremendous heterogeneity of modifications with over 5000 ubiquitinated proteins in cells (Kim et al., Mol Cell 44:325-340 (2011)), and the rapid proteasomal degradation of ubiquitinated proteins.

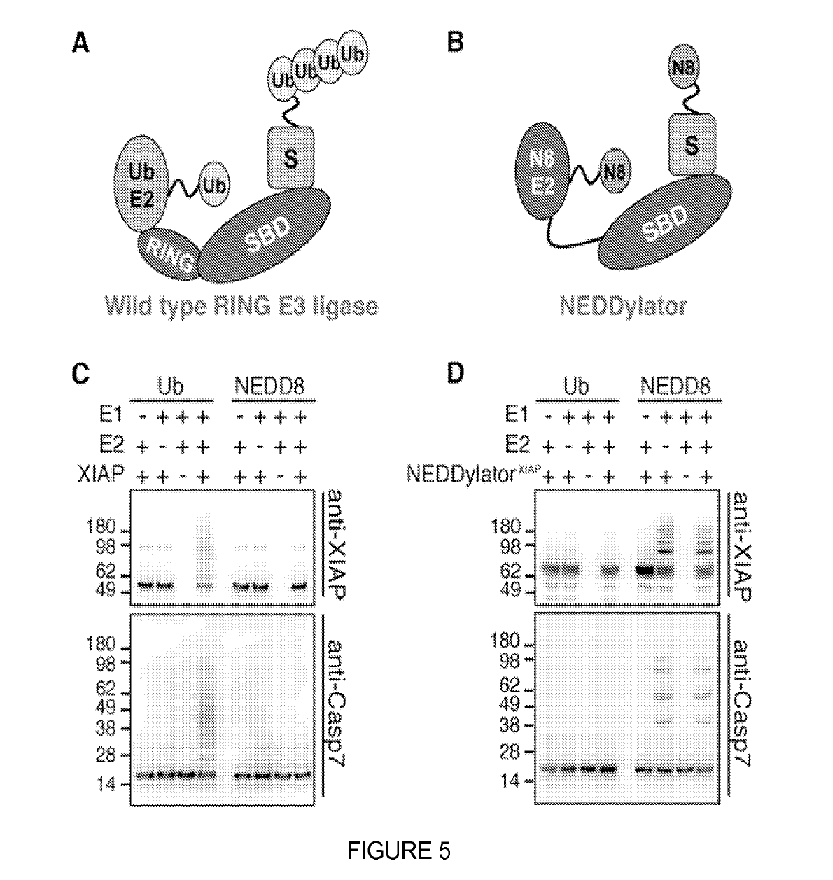

An exemplary catalytic tagging system covalently marks E3 substrates with a rare and stable ubiquitin-like protein, e.g., NEDD8 (see FIG. 5). NEDD8, like ubiquitin, is covalently conjugated to its substrate proteins through lysines. The exemplary catalytic tagging system employs a NEDD8 E1 activating enzyme, a NEDD8 E2 conjugating enzyme, and E3 ligases that provide high signal to noise binding data. NEDD8, as well as other ubiquitin-like proteins, is highly homologous to ubiquitin, but unlike ubiquitin, and has the advantage of having a very limited number of endogenous protein substrates (Xirodimas, Biochem Soc Trans 36:802-806 (2008)). A NEDDylated product is also less complex than poly ubiquitination and resists proteosomal degradation, thus making it a more stable tag. An exemplary catalytic tagging system can additionally covalently mark small molecule targets with stable ubiquitin-like proteins. The exemplary catalytic tagging system permits robust, stable and simple-patterned NEDDylation of bait substrate proteins that are readily purified and identified by mass spectrometry.

### DEFINITIONS

Unless otherwise noted, the technical terms used herein are according to conventional usage as understood by persons skilled in the art. Definitions of common terms in molecular biology may be found in standard texts (e.g. Benjamin Lewin, Genes V, published by Oxford University Press, 1994 (ISBN 0-19854287-9); Kendrew et al. (eds.), The Encyclopedia of Molecular Biology, published by Blackwell Science Ltd, 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The term “bait” as used herein refers to a composition that is fused to one or more other polypeptides, wherein the bait is used to investigate interactions between said bait and one or more substrates of the bait. Non-limiting examples of a bait can include peptides, such as modified E3 ligases, proteins, nucleic acids, carbohydrates, small molecules, such as dasatinib, or other bio-active molecules.

The terms “E3 ubiquitin ligase,” “E3 protein,” “ubiquitin-protein ligase” and “ubiquitin ligase” are used interchangeably herein to refer to ubiquitin ligases which are enzymes that mediate the covalent attachment of ubiquitin or a ubiquitin-like protein to a ubiquitinated substrate. As used herein, these terms also refer to naturally occurring variants of a given E3 ubiquitin ligase.

Non-limiting examples of E3 proteins include: RING ligase, XIAP, cIAP1, human ubiquitin-protein ligase gp78, also known as autocrine motility factor receptor, isoform 2; yeast ubiquitin-protein ligase Doa10; human ubiquitin-protein ligase RNF13; human ubiquitin-protein ligase RNF38; human ubiquitin-protein ligase TEB4; human ubiquitin-protein ligase RNF139 also known as trc8; human ubiquitin-protein ligase RNFx2; human ubiquitin-protein ligase RNF126; human ubiquitin-protein ligase Hrd1; and human ubiquitin-protein ligase MARCH1.

A “modified E3 ligase” is a ligase modified from its naturally occurring form, for example, to prevent the E3 ligase from binding to a ubiquitin E2 ligase. A non-limiting example of a modified E3 ligase is an E3 ligase comprising a substrate binding domain and flexible linker, e.g., a Gly-Gly-Ser-Gly linker, and absent any portion of a RING domain, that is able to bind to an E2 ubiquitin-like conjugating enzyme. The term “modify” or “modifying” and grammatical variations thereof, when used in reference to a composition such as a protein or protein fragment, means that the modified composition deviates from a reference composition. As used herein, modifying can include the addition or removal of protein domains to assist in the construction of a catalytic tagging system, e.g., removing the RING domain of an E3 ligase that under wild-type conditions would be necessary to bind to a ubiquitin E2 ligase to permit the remaining portions of the E3 ligase absent the RING domain to bind to a ubiquitin-like E2 ligase other than a ubiquitin-like E2 ligase, for example, a NEDD8 E2 ligase.

The term “small molecule” refers to a non-peptidic, non-oligomeric organic compound either synthesized in the laboratory or found in nature. Small molecules, as used herein, can refer to compounds that are “natural product-like”, however, the term “small molecule” is not limited to “natural product-like” compounds. Rather, a small molecule is typically characterized in that it contains several carbon-carbon bonds, and has a molecular weight of less than 1500, although this characterization is not intended to be limiting for the purposes of the present invention. Non limiting examples of small molecules that can be used in accordance with the invention described herein are dasatinib, imatinib, nilotinib, thalidomide, lenalidomide, and pomalidomide.

The term “kinase” refers to any enzyme that catalyzes the addition of phosphate groups to a protein residue; for example, serine and threonine kinases catalyze the addition of phosphate groups to serine and threonine residues.

The term “phosphatase” refers to an enzyme capable of removing a phosphate group from a protein, polypeptide, or a peptide by a hydrolytic reaction.

The terms “E2 ubiquitin conjugating enzyme,” “E2 ubiquitin-like conjugating enzyme,” “E2 protein,” “ubiquitin-like conjugating enzyme,” and “ubiquitin conjugating enzyme” are used interchangeably herein to refer to enzymes that interact with ubiquitin or a ubiquitin-like protein and an E3 protein in the ubiquitylation or neddylation or respective process. As used herein, these terms also refer to naturally occurring variants of a given E2 protein and recombinantly prepare variants, for functional fragments, thereof. Non-limiting examples of ubiquitin-like proteins are e.g., NEDD8 E2 (e.g. Ubc12), SUMO E2, ISG15 E2, ATG8 E2, ATG12 E2, and FAT10 E2.

The term “fusion” or “fusing” and grammatical variants thereof, when used in reference to a composition such as a protein or protein fragment, refers to the assembly of two or more protein regions, or fragments thereof, to generate fusions. Fusions can be created by several means, e.g., chemically by coupling, conjugation or cross-linking, either directly or through an intermediate structure; physically by coupling through capture in or on a macromolecular structure; or by molecular biological fusion, through the combination of recombinant nucleic acid molecules that comprise fragments of nucleic acid capable of encoding each of the two, such that a single continuous expression product is produced. An exemplary fusion includes, but is not limited to, a catalytic tagging system described herein having the substrate binding domain of an E3 ligase fused to a ubiquitin-like E2 ligase such as Ubc12. The term “fused” means to create a fusion protein as mentioned above. The term “fusion protein” used herein relates to an artificial proteinaceous construct and means a protein comprising at least two heterologous amino acid sequences which are defined by their origin and/or by special functions. Moreover, the term fusion protein can further include such fusion proteins which also contain non-protein molecule parts such as nucleic acids, sugars, or markers for radioactive or fluorescent labeling.

The term “wild-type conditions” as used herein refers to conditions under which a naturally occurring E3 ligase binds an E2 ubiquitin conjugating enzyme.

The term “substrate binding domain” as used herein refers to an E3 domain capable of binding to a substrate that is capable of being bound to ubiquitin or ubiquitin-like proteins (See, e.g., Liu and Nussinov J. Mol. Biol. 395:1508-23 (2010)).

The term “ubiquitin-like protein” is a protein having high homology to ubiquitin, and which can be covalently conjugated to its substrate proteins through lysines. By way of example, NEDD8, SUMO, ISG15, ATG8, ATG12, and FAT10 are ubiquitin-like proteins.

The term “conjugating” or “to conjugate” or “conjugation” refers to the process of linking, connecting, associating, or any combination thereof, two or more smaller entities, such as protein or protein fragments, to form a larger entity. A non-limiting example as described herein is the conjugation of a ubiquitin-like protein to an E3 ligase substrate.

The term “substrate” as used herein refers to any substrate that is capable of being conjugated or associated with ubiquitin or a ubiquitin-like protein. The substrate can be any naturally occurring or synthetic substrate.

The term “cell extract” is intended to include cell lysate, and culture solution which contains the fusion protein and substrate.

The term “mass spectrometry” or “MS” refers to an analytical technique to identify compounds by their mass. MS refers to methods of filtering, detecting, and measuring ions based on their mass-to-charge ratio, or “m/z”. MS technology generally includes (1) ionizing the compounds to form charged compounds; and (2) detecting the molecular weight of the charged compounds and calculating a mass-to-charge ratio. The compounds may be ionized and detected by any suitable means. A “mass spectrometer” generally includes an ionizer and an ion detector. In general, one or more molecules of interest are ionized, and the ions are subsequently introduced into a mass spectrographic instrument where, due to a combination of magnetic and electric fields, the ions follow a path in space that is dependent upon mass (“m”) and charge (“z”). See, e.g., U.S. Pat. No. 6,204,500, entitled “Mass Spectrometry From Surfaces;” U.S. Pat. No. 6,107,623, entitled “Methods and Apparatus for Tandem Mass Spectrometry;” U.S. Pat. No. 6,268,144, entitled “DNA Diagnostics Based On Mass Spectrometry;” U.S. Pat. No. 6,124,137, entitled “Surface-Enhanced Photolabile Attachment And Release For Desorption And Detection Of Analytes;” Wright et al., Prostate Cancer and Prostatic Diseases 2:264-76 (1999); and Merchant and Weinberger, Electrophoresis 21: 1164-67 (2000. Non-limiting examples are SILAC mass spectrometry and liquid chromatography-tandem mass spectrometry (LC/MS/MS).

The term “apoptosis” refers to the form of eukaryotic cellular death, which is distinct form necrosis, and which includes cytoskeletal disruption, cytoplasmic shrinkage and condensation, expression of phosphatidylserine on the outer surface of the cell membrane and blebbing, resulting in the formation of cell membrane bound vesicles or apoptotic bodies. For a review of apoptotic cell death see, e.g., Utz & Anderson, 2000, Life and death decisions: regulation of apoptosis by proteolysis of signaling molecules, Cell Death Differ., 7:589-602.

The term “isolated” as used herein with respect to nucleic acids, such as DNA or RNA, refers to molecules separated from other DNAs, or RNAs, respectively that are present in the natural source of the macromolecule. Isolated is meant to include nucleic acid fragments which are not naturally occurring as fragments and would not be found in the natural state. The term isolated as used herein also refers to a nucleic acid or peptide that is substantially free of cellular material, viral material, or culture medium when produced by recombinant DNA techniques, or chemical precursors, or other chemicals when chemically synthesized.

The term “recombinant nucleic acid molecule” refers to a non-naturally occurring nucleic acid molecule containing two or more linked polynucleotide sequences. A recombinant nucleic acid molecule can be produced by recombination methods, particularly genetic engineering techniques, or can be produced by a chemical synthesis method. A recombinant nucleic acid molecule can encode a fusion protein, for example, a modified E3 ligase fused to a ubiquitin-like E2 ligase as described herein. The term “recombinant host cell” refers to a cell that contains a recombinant nucleic acid molecule. As such, a recombinant host cell can express a polypeptide from a “gene” that is not found within the native (non-recombinant) form of the cell.

Reference to a polynucleotide “encoding” a polypeptide means that, upon transcription of the polynucleotide and translation of the mRNA produced there from, a polypeptide is produced. The encoding polynucleotide is considered to include both the coding strand, whose nucleotide sequence is identical to an mRNA, as well as its complementary strand. It will be recognized that such an encoding polynucleotide is considered to include degenerate nucleotide sequences, which encode the same amino acid residues. Nucleotide sequences encoding a polypeptide can include polynucleotides containing introns as well as the encoding exons.

An expression control sequence refers to a nucleotide sequence that regulates the transcription or translation of a polynucleotide or the localization of a polypeptide to which it is operatively linked. Expression control sequences are “operatively linked” when the expression control sequence controls or regulates the transcription and, as appropriate, translation of the nucleotide sequence (e.g., a transcription or translation regulatory element, respectively), or localization of an encoded polypeptide to a specific compartment of a cell. Thus, an expression control sequence can be a promoter, enhancer, transcription terminator, a start codon (ATG), a splicing signal for intron excision and maintenance of the correct reading frame, a STOP codon, a ribosome binding site, or a sequence that targets a polypeptide to a particular location, for example, a cell compartmentalization signal, which can target a polypeptide to the cytosol, nucleus, plasma membrane, endoplasmic reticulum, mitochondrial membrane or matrix, chloroplast membrane or lumen, medial trans-Golgi cistemae, or a lysosome or endosome. Cell compartmentalization domains are well known in the art and include, for example, a peptide containing amino acid residues 1 to 81 of human type II membrane-anchored protein galactosyltransferase, or amino acid residues 1 to 12 of the presequence of subunit IV of cytochrome c oxidase (see also Hancock et al., EMBO J. 10:4033-4039, 1991; Buss et al., Mol. Cell. Biol. 8:3960-3963, 1988; and U.S. Pat. No. 5,776,689; each of which is incorporated herein by reference).

The term “operatively linked” or “operably linked” or “operatively joined” or the like, when used to describe chimeric (e.g., fusion) proteins, refer to polypeptide sequences that are placed in a physical and functional relationship to each other. In a most preferred embodiment, the functions of the polypeptide components of the chimeric protein are unchanged compared to the functional activities of the parts in isolation. As used herein, the fusion proteins of the invention can be in a monomeric state, or in a multimeric state (e.g., dimeric).

The term “nucleic acid” refers to deoxyribonucleotides or ribonucleotides and polymers thereof in either single- or double-stranded form, and complements thereof. The term refers to all forms of nucleic acids (e.g., gene, pre-mRNA, mRNA) and their polymorphic variants, alleles, mutants, and interspecies homologs. The term nucleic acid is used interchangeably with gene, cDNA, mRNA, oligonucleotide, and polynucleotide. The term encompasses nucleic acids that are naturally occurring or recombinant.

The term “identical” or “identity” or “percent identity,” or “sequence identity” in the context of two or more nucleic acids or polypeptide sequences that correspond to each other refer to two or more sequences or subsequences that are the same or have a specified percentage of amino acid residues or nucleotides that are the same (e.g., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical” and are embraced by the term “substantially identical.” This definition also refers to, or can be applied to, the compliment of a test sequence. The definition also includes sequences that have deletions and/or additions, as well as those that have substitutions. As described below, the preferred algorithms can account for gaps and the like. Preferably, identity exists for a specified entire sequence or a specified portion thereof or over a region of the sequence that is at least about 25 amino acids or nucleotides in length, or more preferably over a region that is 50-100 amino acids or nucleotides in length. A corresponding region is any region within the reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters. A comparison window includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted (e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologus protein indicates that the protein comprises two or more subsequences that are not found in the same relationship to each other in nature (e.g., a fusion protein).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. Antibodies can be polyclonal or monoclonal, derived from serum, a hybridoma or recombinantly cloned, and can also be chimeric, primatized, or humanized. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH—CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

The phrase “specifically (or selectively) binds” when referring to a protein, nucleic acid, antibody, or small molecule compound refers to a binding reaction that is determinative of the presence of the protein or nucleic acid, such as the differentially expressed genes of the present invention, often in a heterogeneous population of proteins or nucleic acids and other biologics. In the case of antibodies, under designated immunoassay conditions, a specified antibody may bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

### Catalytic Tagging Systems

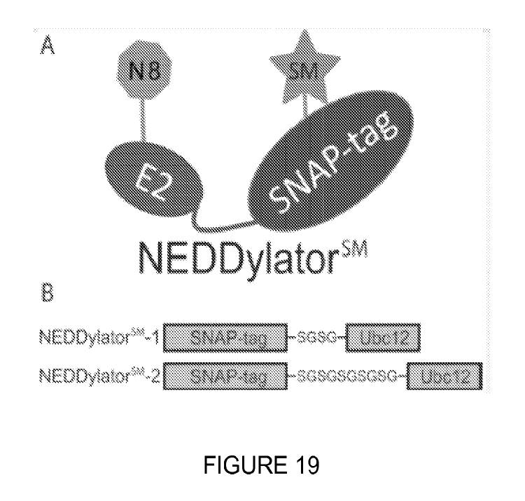

An embodiment includes a catalytic tagging system that is a chimeric protein with NEDD8 E2 (Ubc12) fused to the N-terminus (residues 1-434) of an XIAP E3 ubiquitin ligase fragment that lacks all or part of a RING domain, but retains a substrate binding domain and acts as a bait (e.g., FIG. 5B). Another embodiment includes a catalytic tagging system that is a chimeric protein with NEDD8 E2 (Ubc12) fused to a small molecule used as bait. The small molecule can be fused to the NEDD8 E2 via a SNAP-tag that is associated with the NEDD8 E2 via an amino acid linker, for example, a four amino acid or ten amino acid linker (FIG. 19). Other embodiments include catalytic tagging systems that can use, for example, nucleic acids, peptides, carbohydrates, and other bio-active molecules as baits.

RING domains in E3 ubiquitin ligases function to bind ubiquitin E2s and place them in close proximity to the bound substrate so they can catalyze the transfer of ubiquitin from the E2 to amino groups on the substrate bound to its E3 (FIG. 5A) (see Budhidarmo et al. Trends Biochem. Sci. 37:58-65 (2012). Any portion of the E3 ligase can be removed such that the E3 ligase will be prevented from association with its wild-type E2 binding partner. RING domains include, but are not limited to, polypeptides having an amino acid sequence corresponding to Genbank accession numbers AAD30147, AAD30146, or 6320196. In an embodiment, a catalytic tagging system for human XIAP (NEDDylator^{XIAP}) was engineered to remove the RING domain (residues 435-497) from XIAP to prevent its association with ubiquitin E2 (FIG. 5B). This catalytic tagging system is illustrated to identify numerous IAP substrates targeted for ubiquitin-mediated degradation (see, e.g., Tables 1 and 2).

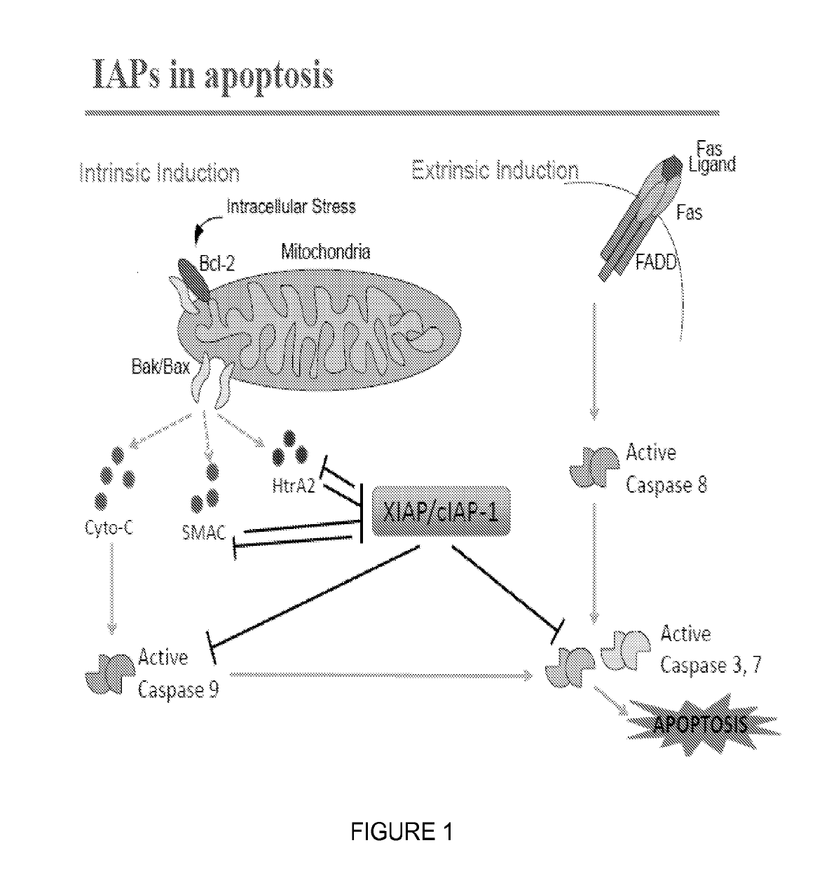

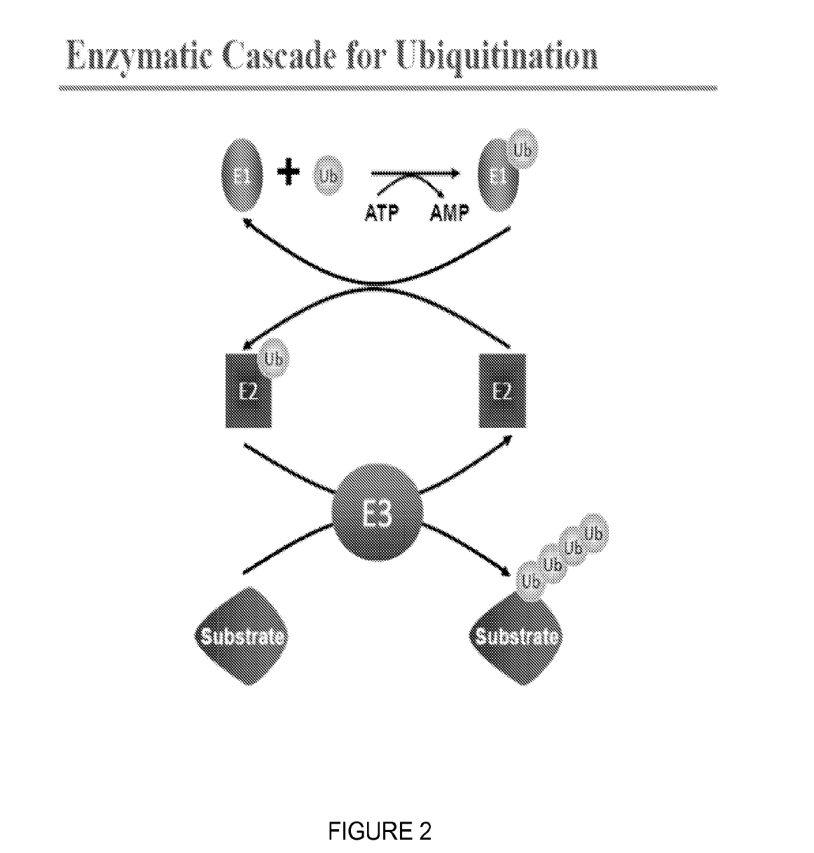

The Inhibitors of Apoptosis Protein (IAPs) are cellular guardians that are critical for controlling pro-death proteins such as caspases, Smac and HtrA2. IAPs are E3 ligases that contain both a RING ubiquitin ligase domain and characteristic baculoviral IAP repeat (BIR) domains that recognize substrates and promote their ubiquitination (Vaux and Silke, Nat Rev Mol Cell Biol 6:287-297 (2005); Vucic et al., Nat Rev Mol Cell Bio 12:439-452 (2011)). See also FIG. 1. One of skill in the art could easily modify the invention as described herein to be used with any E3 ligase that functions to bind substrates for targeted degradation.

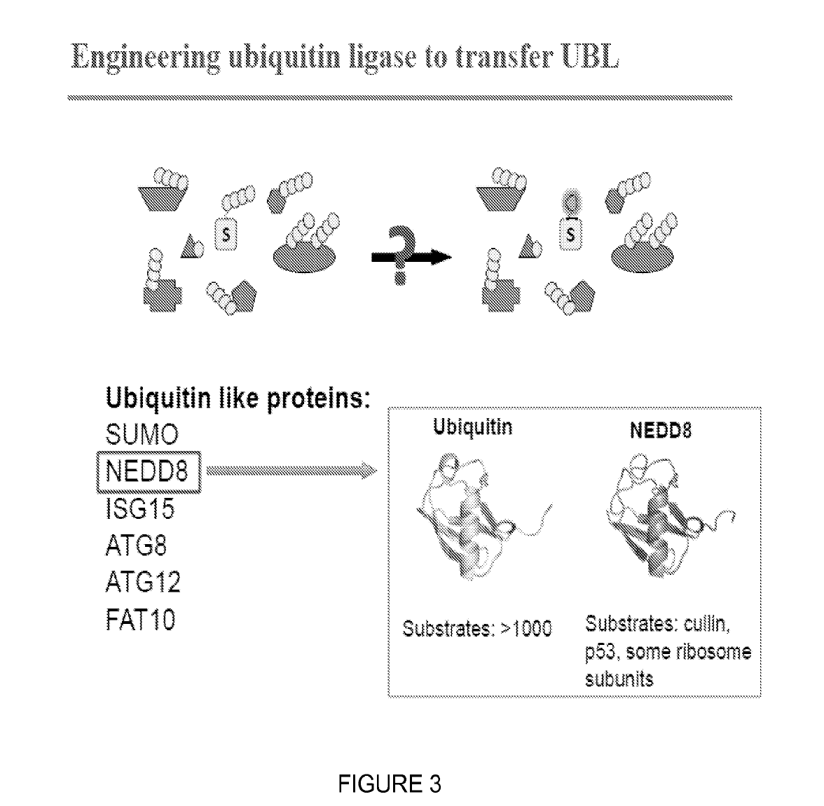

The present invention can be used with any ubiquitin-like protein including, but not limited to, SUMO, NEDD8, ISG15, ATG8, ATG12, and FAT10 (see FIG. 3). Any E2 protein can be used in the NEDDylator system so long as it has the ability to conjugate the chosen ubiquitin-like protein to a target substrate. Exemplary ubiquitin-like E2 conjugating proteins can include, but are not limited to, any E2 (e.g., Ubc12) capable of conjugated a ubiquitin-like protein to a target substrate.

In an embodiment, an exemplary catalytic tagging system comprises an E3 ligase that includes the substrate binding domain, but is devoid of the E3 ligase RING domain. One of skill in the art would recognize that any E3 ligase modification readily envisioned to permit a ubiquitin-like E2 conjugating enzyme to conjugate a ubiquitin-like protein to a selected E3 ligase substrate can be used as described herein.

### Ubiquitination and NEDDylation Assays

The rate or extent of ubiquitination or NEDDylation of a target substrate can be measured in a variety of ways as known in the art. One method is to carry out a ubiquitination or NEDDylation reaction, separating proteins in the reaction mixture by electrophoresis, transferring the separated proteins to a substrate, e.g., Western Blotting, probing the Blot with antibodies against the substrate, and detecting changes in mobility of the substrate that reflect attachment of ubiquitin, NEDD, or any other ubiquitin-like protein, to the substrate. Other methods of measuring ubiquitination can be used, including without limitation immunologically based assays (ELISA, immunoprecipitation), mass spectrometry, electromagnetic spectrum spectroscopic methods, chromatographic methods, ubiquitination or NEDDylation using detectably labeled ubiquitin or a detectably labeled ubiquitin-like protein or a detectably labeled NEDD. Other approaches apparent to those of skill in the art can be used to detect ubiquitination or NEDDylation. For example, in a plate assay, fluorescein-tagged ubiquitin or a ubiquitin-like protein can be detected directly using a fluorescence plate reader, biotin-tagged ubiquitin or a ubiquitin-like protein can be detected using labeled strepavidin, and epitope-tagged ubiquitin or an epitope-tagged ubiquitin-like protein can be detected in immunoassays using antibodies against the selected epitope-tag.

A large number of assay formats can be used for the ubiquitination and NEDDylation assays. For example, the assay components can be in solution, or one or more components can be immobilized. A ubiquitination or NEDDylation assay can also, for example, be carried out by adding ubiquitin or a ubiquitin-like protein to a well, tube, or chamber. For illustration, when a target substrate is immobilized on a surface (e.g., a microwell plate, Sepharose beads, magnetic beads) and incubated with a ligase reaction mix including the components of the reaction.

### Detecting Bait Substrates

Embodiments of the present invention can globally identify bait substrates through a variety of known mechanisms, including but not limited to, stable isotope labeling by amino acid in cell culture (SILAC)-based mass spectrometry.

Stable isotope labeling by amino acid (SILAC) can be used to assay relative concentrations of proteins of cells grown in culture. SILAC incorporates a label into proteins for mass spectrometric (MS)-based proteomics. SILAC relies on metabolic incorporation of a “light” or “heavy” form of an amino acid into proteins.

In a SILAC assay, two groups of cells are grown in culture media that are essentially identical except in one respect: one media contains a “light” and the other a “heavy” form of a particular amino acid. Thus, parallel cultures are typically grown where one set of cultures is grown in media containing an isotopically-labeled amino acid (e.g. ¹⁵N-Arg) and the other culture set is grown in conventional media thereby allowing an investigator to challenge one set of cultures with an external stimulus to monitor the relative changes in expression. With each cell doubling the cell population replaces at least half of the original form of the amino acid, eventually incorporating 100% of a given “light” or “heavy” form of the amino acid. Thus, when the labeled analog of an amino acid is supplied to cells in culture instead of the natural amino acid, it is incorporated into all newly synthesized proteins. After a number of cell divisions, each instance of the particular amino acid will be replaced by its isotope-labeled analog. Because there is little chemical difference between the labeled amino acid and the natural amino acid isotopes, the cells behave like the control cell population grown in the presence of normal amino acids. SILAC methods are disclosed for example, in U.S. Pat. No. 6,391,649.

Using SILAC, quantitation by mass spectrometry (MS) is performed by measuring the relative peak intensities of the heavy-labeled and the light-labeled isoforms of the peptides. The difference in the mass of the isotope in each cell pool results in two distinct, closely spaced peaks for each protein or peptide actively produced by the samples in the mass spectrum. One peak corresponds to a protein or peptide from a protein from the cell pool with the normal abundance of isotopes. The other peak corresponds to a protein or peptide from the cell pool enriched in one or more of the isotopes. A ratio is computed between the peak intensities of at least on pair of peaks in the mass spectrum. The relative abundance of the protein in each sample may be determined based on the computed ratio. The protein may be identified by the mass-to-charge ratios of the peaks in the mass spectrum, as well as by other means known in the art.

In a typical mass spectrometer, a probe with a marker is introduced into an inlet system of the mass spectrometer. The analyte is then desorbed by a desorption source such as a laser, fast atom bombardment, or high energy plasma. The generated desorbed, volatilized species consist of preformed ions or neutrals that are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of a marker or other substances will typically involve detection of signal intensity. This, in turn, reflects the quantity and character of a marker bound to the probe. Mass spectrometry can include liquid chromatography-tandem mass spectrometry (LC-MS/MS) gas phase ion spectrometry, laser desorption mass spectrometry, tandem mass spectrometry, electrospray mass spectrometry, Surface-Enhanced Laser Desorption/Ionization (“SELDI”) mass spectrometry, or Matrix-Assisted Laser Desorption Ionization-time of Flight Mass Spectrometry (MALDI-TOF MS).

Bait substrates can also be detected by use of other methods known in the art. For example, a substrate can be immobilized on a biochip array and subjected to laser ionization to detect the molecular weight of the substrates. Analysis of the substrates is, for example, by molecular weight of the one or more markers against a threshold intensity that is normalized against total ion current. Substrates can be detected by other known methods in the art, which include but are not limited to, surface plasmon resonance, ellipsometry, and atomic force microscopy.

### Cloning and Recombinant Protein Expression and Purification

Embodiments of the present invention include assays and compositions relating to protein engineering necessary for the construction of exemplary catalytic tagging systems. To obtain high level expression of a cloned gene or genome, one typically subclones a nucleic acid into an expression vector that contains a strong promoter to direct transcription, a transcription/translation terminator, and if for a nucleic acid encoding a protein, a ribosome binding site for translational initiation. Suitable bacterial promoters are well known in the art and described (e.g., in Sambrook et al., and Ausubel et al., supra. Bacterial expression systems for expressing the protein are available in, e.g., E. coli, Bacillus sp., and Salmonella (Palva et al., Gene 22:229-235 (1983)); Mosbach et al., Nature 302:543-545 (1983)). Kits for such expression systems are commercially available. Eukaryotic expression systems for mammalian cells, yeast, and insect cells are well known in the art and are also commercially available. Retroviral expression systems can be used in the present invention.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function. Heterologous refers to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

In addition to the promoter, the expression vector typically contains a transcription unit or expression cassette that contains all the additional elements required for the expression of the nucleic acid in host cells. A typical expression cassette thus contains a promoter operably linked to the nucleic acid sequence encoding the nucleic acid of choice and signals required for efficient polyadenylation of the transcript, ribosome binding sites, and translation termination. Additional elements of the cassette can include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region can be obtained from the same gene as the promoter sequence or can be obtained from different genes.

The particular expression vector used to transport the genetic information into the cell is not particularly critical. Any of the conventional vectors used for expression in eukaryotic or prokaryotic cells can be used. Standard bacterial expression vectors include plasmids such as pBR322 based plasmids, pSKF, pET23D, PGEX6P-1, pRSFDuet, and fusion expression systems such as MBP, GST, and LacZ. Epitope tags can also be added to recombinant proteins to provide convenient methods of isolation, e.g., c-myc. Sequence tags can be included in an expression cassette for nucleic acid rescue. Markers such as fluorescent proteins, green or red fluorescent protein, 13-gal, CAT, and the like can be included in the vectors as markers for vector transduction.

Expression vectors containing regulatory elements from eukaryotic viruses are typically used in eukaryotic expression vectors, e.g., SV40 vectors, papilloma virus vectors, retroviral vectors, and vectors derived from Epstein-Barr virus. Other exemplary eukaryotic vectors include pMSG, pAV009/A+, pMT010/A+, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can also be regulated using inducible promoters. With inducible promoters, expression levels are tied to the concentration of inducing agents, such as tetracycline, by the incorporation of response elements for these agents into the promoter. Generally, high level expression is obtained from inducible promoters only in the presence of the inducing agent; basal expression levels are minimal.

Vectors can have a regulatable promoter, e.g., tet-regulated systems and the RU-486 system (see, e.g., Gossen & Bujard, PNAS 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)). These impart small molecule control on the expression of the candidate target nucleic acids. This beneficial feature can be used to determine that a desired phenotype is caused by a transfected cDNA rather than a somatic mutation.

Some expression systems have markers that provide gene amplification such as thymidine kinase and dihydrofolate reductase. Alternatively, high yield expression systems not involving gene amplification are also suitable, such as using a baculovirus vector in insect cells, with a sequence of choice under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, as any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (1983)).

Any of the well-known procedures for introducing foreign nucleotide sequences into host cells can be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing proteins and nucleic acids as described herein.

After the expression vector is introduced into the cells, the transfected cells are cultured under conditions favoring expression of the protein of choice, which is recovered from the culture using standard techniques known in the art. See, e.g., Freshney et al., Culture of Animal Cells, A Manual of Basic Technique (3rd ed. 1994), and the references cited therein for a discussion of cell culture conditions and how to isolate and culture cells from patients. Conditions for pancreatic cells in particular have been described (Lehmann et al. Diabetes 56:594-603 (2007) and King et al. Diabetes 56:2312-18 (2007)). In general, the cell culture environment includes consideration of such factors as the substrate for cell growth, cell density and cell contract, the gas phase, the medium, and temperature. Incubation of cells is generally performed under conditions known to be optimal for cell survival.

Plastic dishes, flasks, or roller bottles may be used to culture cells according to the methods of the present invention. Suitable culture vessels include, for example, multi-well plates, Petri dishes, tissue culture tubes, flasks, roller bottles, and the like.

Cells are grown at optimal densities that are determined empirically based on the cell type. Cultured cells are normally grown in an incubator that provides a suitable temperature, e.g., the body temperature of the animal from which is the cells was obtained, accounting for regional variations in temperature. Generally, 37° C. is the preferred temperature for cell culture. Most incubators are humidified to approximately atmospheric conditions.

Defined cell media are available as packaged, premixed powders or presterilized solutions. Examples of commonly used media include YT, MEM-a, DME, RPMI 1640, DMEM, Iscove's complete media, or McCoy's Medium (see, e.g., GibcoBRL/Life Technologies Catalogue and Reference Guide; Sigma Catalogue). Typically, MEM-a or DMEM are used in the methods of the invention. Defined cell culture media are often supplemented with 5-20% serum, typically heat inactivated serum. Cell culture can be further supplemented with selection compounds, including but not limited to, ampicillin. The culture medium is usually buffered to maintain the cells at a pH preferably from about 7.2 to about 7.4. Other supplements to the media typically include, e.g., antibiotics, amino acids, and sugars, and growth factors.

Proteins of the present invention can be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant protein is being purified. For example, proteins having established molecular adhesion properties can be reversible fused to the protein. With the appropriate ligand or substrate, a specific protein can be selectively adsorbed to a purification column and then freed from the column in a relatively pure form. The fused protein is then removed by enzymatic activity. Finally, protein can be purified using immunoaffinity columns. Recombinant protein can be purified from any suitable source, include yeast, insect, bacterial, and mammalian cells.

Recombinant proteins can be expressed and purified by transformed bacteria in large amounts, typically after promoter induction; but expression can be constitutive. Promoter induction with IPTG is one example of an inducible promoter system. Bacteria are grown according to standard procedures in the art. Fresh or frozen bacteria cells are used for isolation of protein.

Proteins expressed in bacteria can form insoluble aggregates (“inclusion bodies”). Several protocols are suitable for purification of protein inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgC12, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies can be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation can occur upon removal (by dialysis, for example) or dilution of the denaturant, allowing re-formation of immunologically and/or biologically active protein. Other suitable buffers are known to those skilled in the art. Human proteins are separated from other bacterial proteins by standard separation techniques, e.g., with Ni-NTA agarose resin.

Alternatively, it is possible to purify recombinant protein from bacteria periplasm. After lysis of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO4 and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

Solubility fractionation can be used as a standard protein separation technique for purifying proteins. As an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

The molecular weight of the protein can be used to isolate it from proteins of greater and lesser size using ultrafiltration through membranes of different pore size (e.g., Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

The protein can also be separated from other proteins on the basis of its size, net surface charge, hydrophobicity, and affinity for ligands or substrates using column chromatography. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

### Therapeutic Methods

An embodiment of the present invention relates to therapeutic methods relating to the identification of substrates that induce activation of apoptosis. The present invention thus includes therapeutic methods useful for the modulation of apoptosis or neoplastic disease, comprising administering to a subject in need of such treatment an effective amount of one or more substrates identified by an exemplary catalytic tagging system described herein, or a pharmaceutically acceptable salt or prodrug of one or more substrates as described herein.

Pharmaceutical compositions comprising the substrates described herein within the scope of the present invention can also contain other compounds, which can be biologically active or inactive. For example, one or more immunogenic portions of other antigens can be present, either incorporated into a fusion polypeptide or as a separate compound, within a composition or vaccine. Polypeptides can, but need not be, conjugated to other macromolecules as described, for example, within U.S. Pat. Nos. 4,372,945 and 4,474,757. Pharmaceutical compositions and vaccines can generally be used for prophylactic and therapeutic purposes.

Formulations suitable for oral administration can consist of (a) liquid solutions, such as an effective amount of the substrate suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (e.g., they can be “nebulized”) to be administered via inhalation. Aerosol formulations can be placed into pressurized acceptable propellants, such as dichlorodifluoromethane, propane, nitrogen, and the like.

Formulations suitable for parenteral administration, for example, by intravenous, topical, subcutaneous, transcutaneous, transdermal, intramuscular, oral, intra-joint, parenteral, intra-arteriole, intradermal, intraventricular, intracranial, intraperitoneal, intralesional, intranasal, rectal, vaginal, or by inhalation. Administration can be targeted directly to pancreatic tissue, e.g., via injection, can include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of commends can be presented in unit-dose or multi-dose sealed containers, such as ampules and vials.

Such compositions can also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention can be formulated as a lyophilizate. Compounds can also be encapsulated within liposomes using well known technology. Injection solutions and suspensions can be prepared from sterile powders, granules, and tablets of the kind previously described.

The dose administered to a subject, in the context of the present invention should be sufficient to affect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular subject.

Pharmaceutically acceptable carriers are determined in part by the particular composition being administered (e.g., nucleic acid, protein, modulatory compounds or transduced cell), as well as by the particular method used to administer the composition. Accordingly, there are a wide variety of suitable formulations of pharmaceutical compositions of the present invention (see, e.g., Remington's Pharmaceutical Sciences, 17th ed., 1989).

### Kits

The invention provides kits for practicing the assays described herein. Kits for carrying out the assays of the invention typically include a modified E3 ligase wherein said modified E3 ligase comprises a substrate binding domain fused to an E2 ubiquitin-like conjugating enzyme that modifies an E2 ubiquitin-like protein other than ubiquitin.

### EXAMPLES

The invention herein described is further illustrated in the following examples, which are provided by way of illustration and are not intended to be limiting.

### Example 1

### Engineering a Catalytic Tagging System for Tagging IAP Substrates

The RING domains in E3 ubiquitin ligases function to bind ubiquitin E2s and place them in close proximity to the bound substrate so they can catalyze the transfer of ubiquitin from the E2 to amino groups on the substrate bound to its E3 (FIG. 5A). A catalytic tagging system for human XIAP (NEDDylator^{XIAP}) was engineered by removing the RING domain (residues 435-497) from XIAP to prevent its association with ubiquitin E2 (FIG. 5B). The N-terminus (residues 1-434) of XIAP was then fused via a flexible Gly-Gly-Ser-Gly linker to the NEDD8 E2, Ubc12. This construct was thus designed to ablate the ability to ubiquitinate, but empower the ability to NEDDylate ubiquitin ligase substrates.

### Cloning and Recombinant Protein Expression and Purification

The catalytic tagging systems were generated with overlap extension PCR, including NEDDylator^{XIAP}, NEDDylator^{cIAP1}, the FKBP-XIAP fusions and FRB-Ubc12 fusions. These were cloned into PGEX6P-1 vector (GE Heathcare) that has an N-terminal glutathione S-transferase (GST) fused in frame with the target gene. Each NEDDylator was expressed in E. Coli BL21 (DE3) cells (Stratagene). Cells were grown in 2xYT media supplemented with 200 μg/ml ampicillin at 37° C. to an OD_{600 nm} around 0.8. Overexpression of the NEDDylator was induced with 0.6 mM IPTG at 16° C. overnight. Cells were harvested, resuspended in 50 mM Tris pH 8.0, 200 mM NaCl, 5 mM DTT and lysed by microfluidization (Microfluidics). The cell lysates were clarified by centrifugation at 48,000×g for 30 min at 4° C. and the soluble fractions were mixed with 1 ml pre-equilibrated glutathione sepharose 4B (GE Healthcare) and rocked at 4° C. for one hour. The beads were washed with cold lysis buffer then transferred to poly-prep chromatography column (GE healthcare) for elution. The GST fused protein was eluted with 10 mM reduced glutathione in lysis buffer. PreScission protease was added to the elution at 1:500 by weight to allow the cleavage of GST tag at 4° C. overnight. Free GST was removed by gel filtration size exclusive chromatography with Superdex 200 column (GE Healthcare).

His₆-Biotin-NEDD8 (HB-NEDD8) was generated by PCR-based gene synthesis (Hoover and Lubkowski, Nucleic Acids Res. 30:e43 (2002)) and cloned into PGEX6P-1 vector. The biotin tag is a bacterially derived 75 amino acid signal peptide that is efficiently modified by biotin in cells (Tagwerker et al., Mol. Cell Proteomics 5:737-748 (2006); Tirat et al., Int. J. Biol. Macromol. 39:66-76 (2006)). Bacterial biotin ligase birA was cloned into pRSFDuet vector (Novagen). HB-NEDD8 and birA plasmids were co-transformed into BL21 cells and cells were grown under 200 μg/ml ampicillin and 50 μg/ml kanamycin selection. Protein overexpression was induced with 0.6 mM IPTG in the medium supplemented with 50 μM biotin. GST fused HB-NEDD8 was purified as described above. Protein purity and biotin modification of HB tag was confirmed by electrospray ionization mass spectrometry on an LCT Premier Mass Spectrometer (Waters).

### In Vitro Ubiquitination and NEDDylation Assays

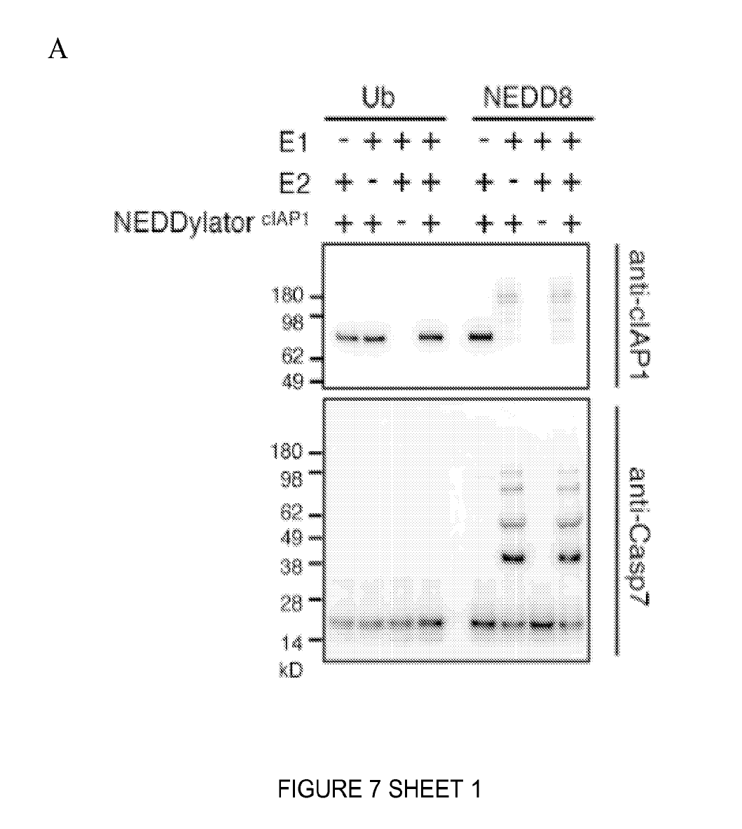

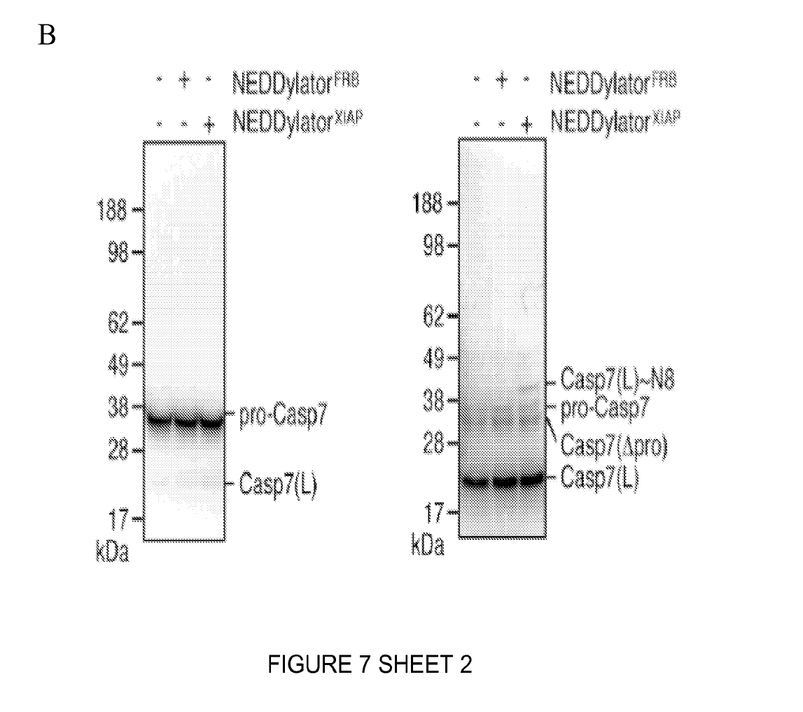

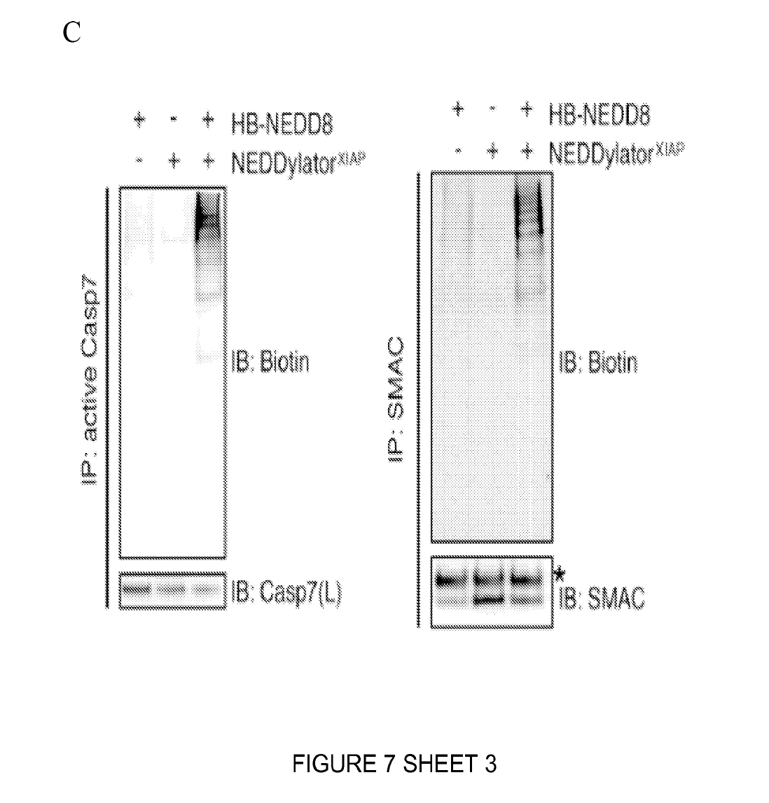

Ubiquitination and NEDDylation were assayed at 37° C. for 1 hour with purified recombinant proteins in a 20 μl reaction volume with 50 mM Tris pH8.0, 150 mM NaCl, 10 mM MgCl₂, 5 mM ATP and 0.1 mM DTT. In FIG. 5 and FIG. 7A, the ubiquitination reactions contain 50 nM UBE1 (Boston Biochem), 200 nM UbcH5b (Boston Biochem), 200 nM NEDDylator, 200 nM active caspase-7 (purified as previously described in (Wolan et al., Science 326:853-858 (2009)), 50 μM ubiquitin (Boston Biochem) and 1 mg/ml bovine serum albumin (BSA, Sigma); the NEDDylation reactions contain 50 nM GST-NAE1, 200 nM Ubc12, 200 nM NEDDylator, 200 nM active caspase-7, 10 μM HB-NEDD8 and 1 mg/ml BSA. All the reactions were stopped by addition of 4×LDS loading buffer (Invitrogen). Caspase-7 and NEDDylator were detected by western blotting using anti-caspase-7 (Cell Signaling, 9494), anti-XIAP (Cell Signaling, 2045) and anti-cIAP1 (Cell Signaling 4952) antibodies.

### In Vivo Ubiquitination and NEDDylation Assays

All mammalian cells used for this study were maintained in the optimized medium as suggested by ATCC. Jurkat cells were harvested and resuspended in 40 million/ml. 300 μl of cells were added to Gene Pulser (BIO-RAD) cuvette (0.4 cm) and electroporated at 250V for 35 mS. HeLa cells were transfected with Lipofectamine 2000 (Invitrogen). Cells were lysed either in M-PER mammalian protein extraction buffer (Thermo) or NP40 buffer (50 mM HEPES pH 7.4, 150 mM NaCl, 1% NP40), supplemented with protease inhibitor cocktail (Thermo). 10% SDS-polyacrylamide gel (PAGE) combined with MOPS running buffer was used to separate full length PGAM5 from the truncated form for immuno-blotting analysis. 4-12% SDS-PAGE gels were generally used for all other analysis. Immuno-precipitation with anti-Myc tag antibody (cell signaling) was performed with a standard procedure using manufacture's instructions. For cytosolic protein extraction, approximately 1×107 Jurkat cells were harvested and washed with cold PBS, resuspended in 250 mM sucrose, 20 mM HEPES pH 7.4, 10 mM KCl, 1.5 mM EGTA, 1.5 mM EDTA, 1 mM MgCl2, 1 mM DTT, and cocktail of protease inhibitors, disrupted by 35 stokes with the glass Dounce and a tight pestle (type B), and then spun at 20,000 g for 15 min at 4° C. The supernatants were considered to be the cytosolic fraction.

### Results

To test this design using purified components, the ability of the native XIAP ubiquitin ligase and the NEDDylator^{XIAP} to transfer either a ubiquitin or a His-biotin tagged NEDD8 (HB-NEDD8) to a known substrate, caspase-7, was compared (Choi et al., J. Biol. Chem 284:12772-12782 (2009); Gray et al., Cell 142:637-646 (2010); Schile et al., Genes Dev. 22:2256-2266 (2008); Suzuki et al., PNAS 98:8662-8667 (2001b)), or to XIAP itself. As expected, wild type XIAP produces multiple ubiquitinated species of caspase-7 and itself, but does not NEDDylate either protein (FIG. 5C). In sharp contrast, the NEDDylator^{XIAP} robustly attaches the tagged NEDD8 to itself and caspase-7 without the need for the NEDD8 E2, and does not ubiquitinate caspase-7 or itself (FIG. 5D).

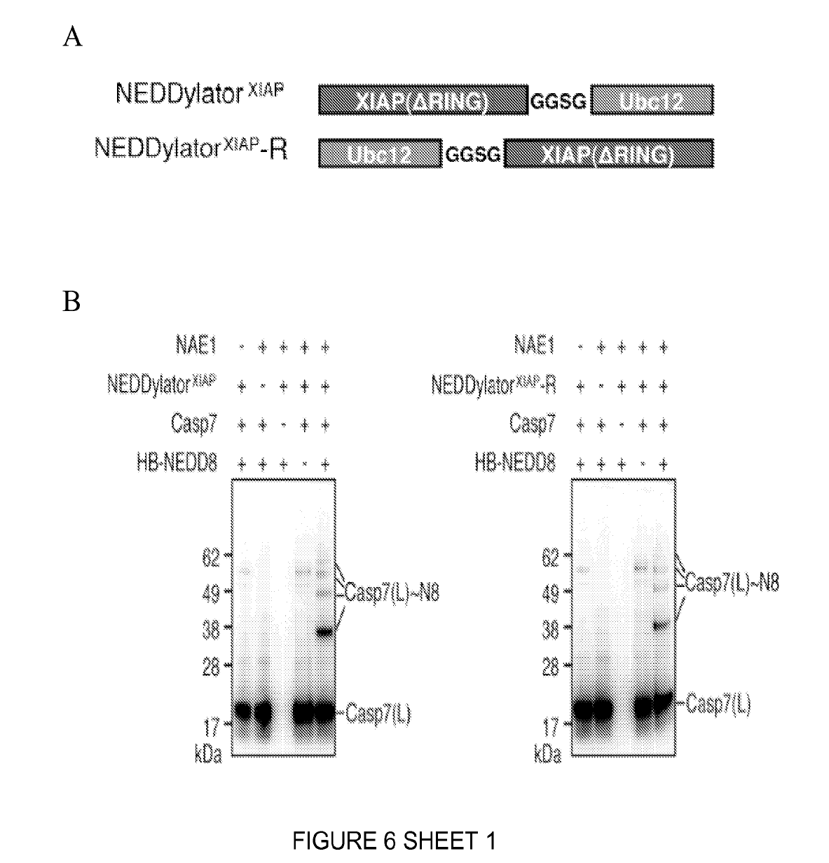

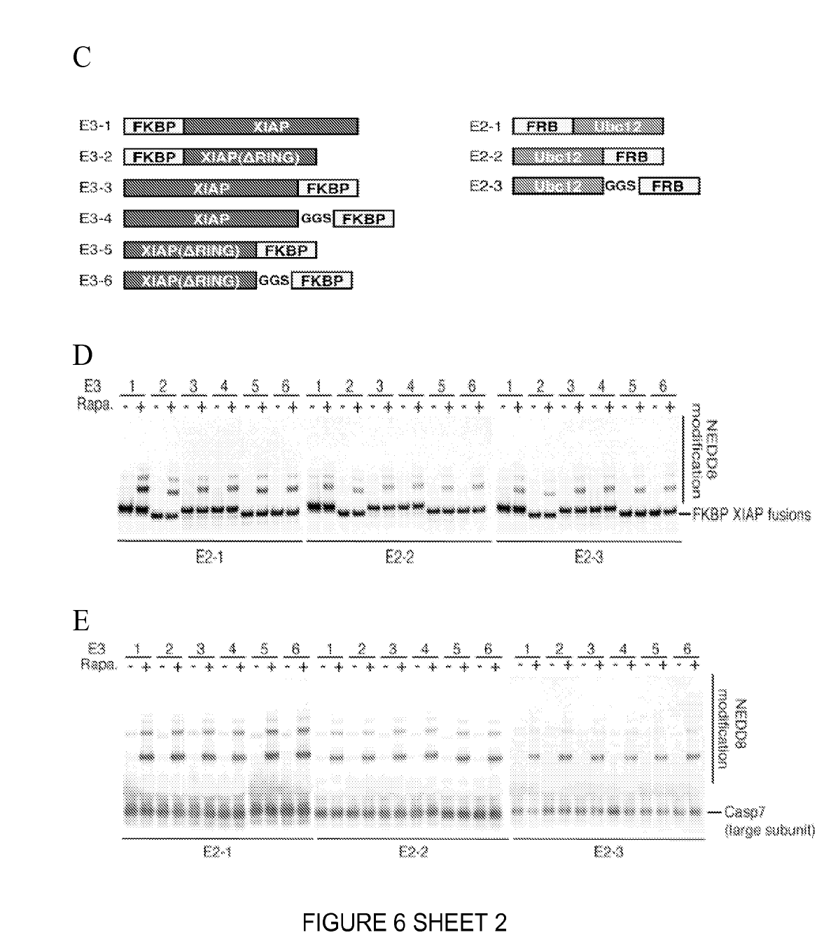

A number of other fusion designs were tested to evaluate the tolerances and flexibility of the NEDDylator^{XIAP} for efficient NEDDylation. To test how orientation affects the NEDDylation, NEDDylator^{XIAP} was compared with a reversed version NEDDylator^{XIAP}-R, in which XIAP is fused to the C-terminal of Ubc12 (FIG. 6A). Similar NEDD8 modifications of caspase-7 were observed between the two constructs (FIG. 6B). To further evaluate the tolerances and to see if the NEDDylator^{XIAP} would be amenable to small molecule conditional induced activation, a split-NEDDylator^{XIAP} was produced in which FKBP was fused to XIAP and FRB separately fused to Ubc12. Only in the presence of rapamycin, which induces heterodimerization of FKBP and FRB (Banaszynski et al., J. Am Chem Soc. 127:4715-4721 (2005)), did the split-NEDDylator^{XIAP} robustly NEDDylate caspase-7. Six FKBP-XIAP and three FRB-Ubc12 fusions were generated with various linkers, potentially introducing different flexibility and orientation between XIAP and Ubc12 (FIG. 6C). Upon rapamyacin induction, all 18 combinations showed NEDDylation efficiencies that were virtually indistinguishable for two known XIAP substrates, caspase-7 and XIAP itself (FIG. 6D, E).

cIAP1 is believed to have both pro-inflammatory and apoptotic roles (Fulda and Vucic, Nat Rev Drug Discov. 11:109-124 (2012)). Its substrates were compared with those of XIAP. The NEDDylator for cIAP1 was generated by removing the C-terminal RING domain of cIAP1 (residues 556-618) and fusing it to Ubc12. The NEDDylator^{cIAP1} showed robust NEDDylation of caspase-7 (FIG. 7A). These results show that the NEDDylator for two different E3 ligases tolerates a wide range of different domain orientations without affecting their abilities to tag target proteins. These data are consistent with the highly flexible nature and structure known for ubiquitin E2-E3 ligase complexes. Such a flexible system evolved to handle a multitude of substrates that vary in size and composition (Duda et al., Cell 134:995-1006 (2008); Zhuang et al., Mol. Cell 36:39-50 (2009)).

The NEDDylator^{XIAP} was next tested in cells and cell lysates. The N-terminal acetylation of Ubc12 is important for specific NEDDylation of cullins (Scott et al., Science 334:674-678 (2011)). Thus, the NEDDylator^{XIAP} having the XIAP fused to the N-terminus of Ubc12 was used to mask the N-terminus of Ubc12 and thereby reduce the background from cullin NEDDylation. Apoptotic Jurkat cell lysates were generated by addition of staurosporin (STS) so that both procaspase-7 (a non-substrate) and active caspase-7 (a known substrate) were present. Addition of the NEDDylator^{XIAP} and free HB-NEDD8 induced NEDD8 modification of mature caspase-7 but not procaspase-7 as expected (FIG. 7B). The NEDDylator was further tested in Jurkat cells, which were transiently transfected with NEDDylator^{XIAP} and HB-NEDD8. Immuno-blotting showed NEDD8 modifications of the known XIAP substrates, caspase-7 and SMAC are significantly increased in the presence of NEDDylator^{XIAP} (FIG. 7C).

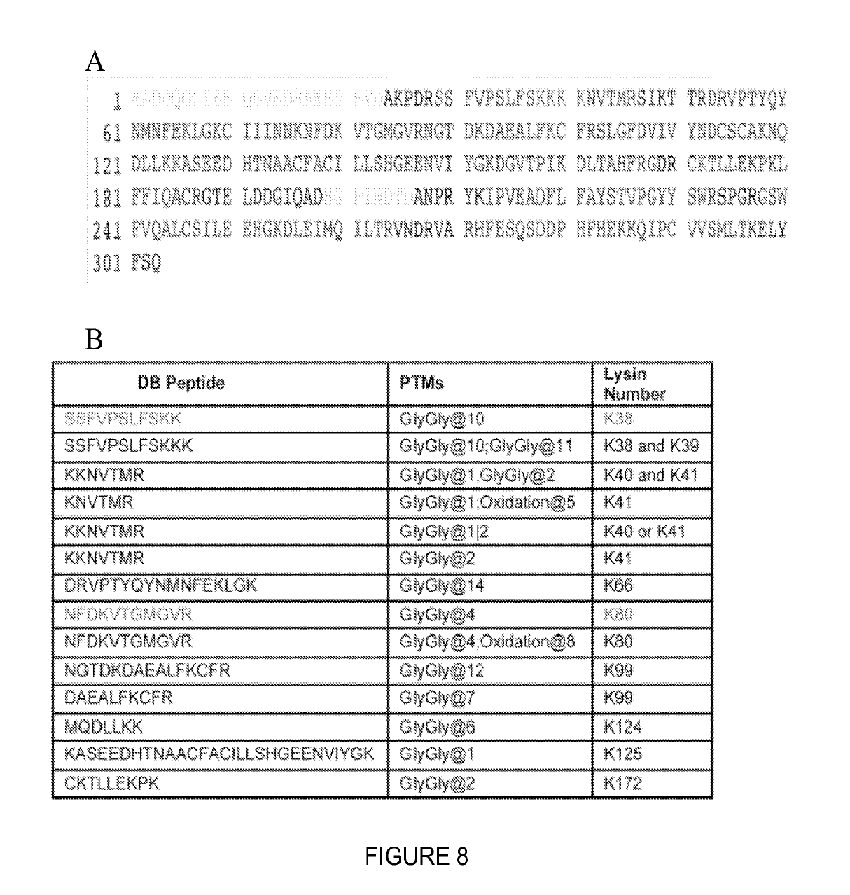

It was next determined which lysines were modified on caspase-7 by the wild type XIAP or the NEDDylator^{XIAP}. Two lysines (K38 and K80) were identified out of 25 on caspase-7 that can be both ubiquitinated by XIAP and modified with NEDD8 by the NEDDylator^{XIAP} (FIG. 8). These data suggest that catalytic tagging system exemplified herein mediated NEDDylation that resembles native ubiquitination and indicates that fusion of the substrate binding domain of XIAP to Ubc12 in the NEDDylator^{XIAP} did not bias the sites that were modified.

### Discussion

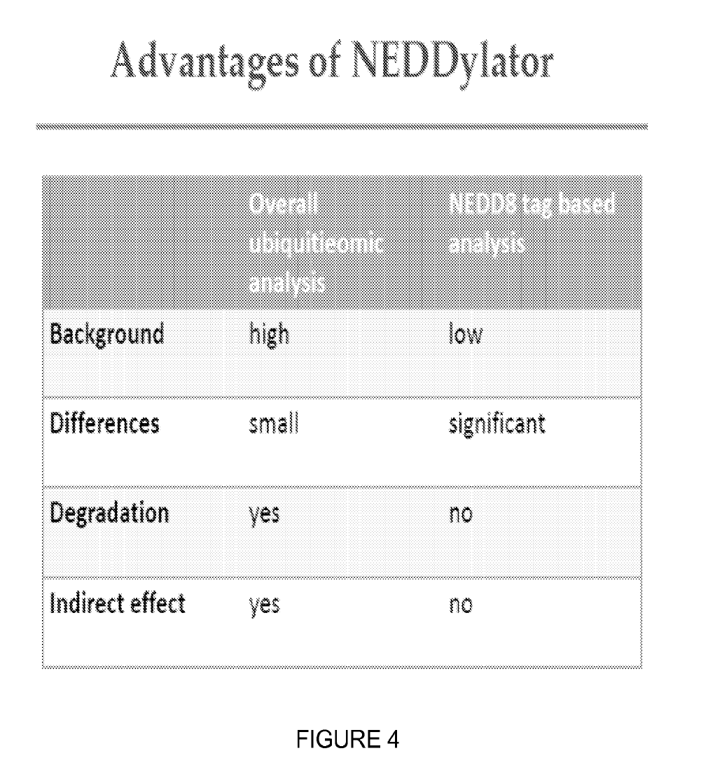

Most ubiquitin substrate identification approaches involve hypothesis-driven candidate approaches, which are slow and heavily biased. Recently, more global substrate identification approaches have been reported using in vitro generated protein microarrays (Gupta et al., Mol. Syst. Biol 3:116 (2007)), quantitative mass spectrometry (Emanuele et al., Cell 147:459-474 (2011); Ota et al., Genes Cells 13:1075-1085 (2008)), or global protein stability profiling (GPS) (Emanuele et al., Cell 147:459-474 (2011); Yen and Elledge, Science 322:923-929 (2008)). Provided herein is an unbiased positive-enrichment method that has several advantages to identifying substrates for specific E3 ubiquitin ligases (see FIG. 4). Unlike microarrays, the substrates identified are from native cellular samples and thus contain resident native protein complexes and post-translational modifications. Second, the catalytic tagging system-based positive enrichment coupled with the dual affinity HB-NEDD8 handle provides a very clean sample that is highly amenable to quantitative mass spectrometry. In addition, modification by the catalytic tagging systems does not allow ubiquitination and provides extremely high signal to noise as shown by the SILAC experiments that greatly simplified the data processing. Third, the NEDDylated product is less complex than poly ubiquitination and does not get degraded by the proteasome making it a more stable mark. Lastly, the method is relatively simple to apply and involves only a single genetically encoded enzyme coupled with mass spectrometry that should be easily adapted by other researchers. The fact that the orientation and a variety of split-constructs were all viable catalytic tagging systems speaks further to the robustness and flexibility of the system.

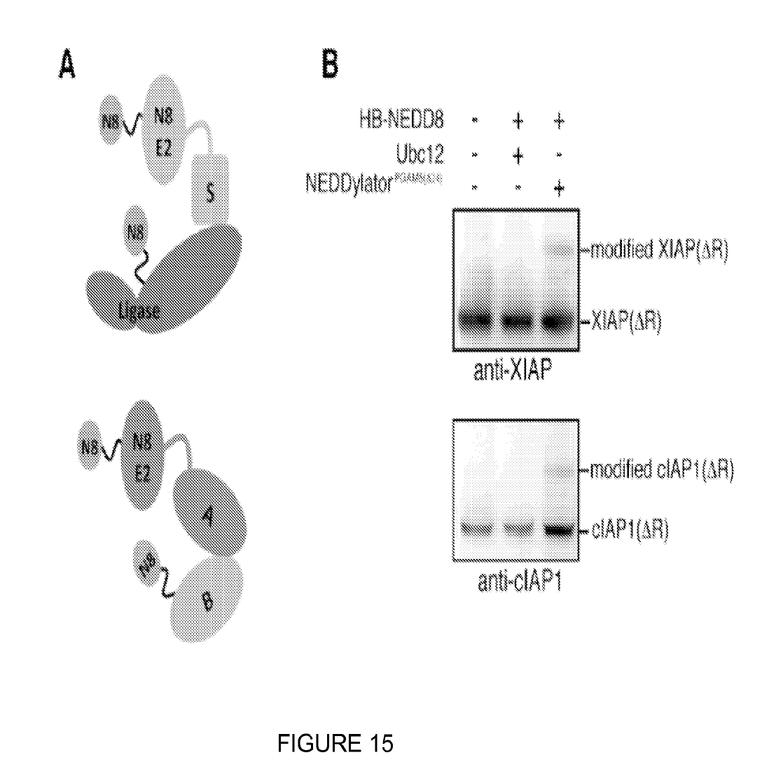

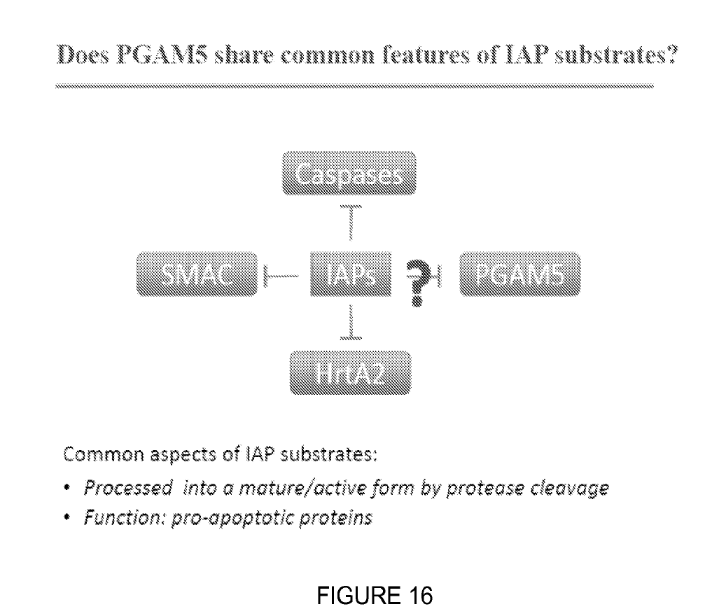

The catalytic tagging strategy can be used to identify other protein-protein interaction partners, by attaching Ubc12 to the bait portion of the protein. As one test described in Example 3 below, the NEDDylator^{PGAM5(Δ24)} was made by fusing Ubc12 to the C-terminus of PGAM5(Δ24) and showed both XIAP and cIAP1 were modified by NEDDylator^{PGAM5(Δ24)} without the need for the RING domain (FIG. 15). This further validates the interactions between PGAM5(Δ24) and IAP proteins, and suggests that the catalytic tagging systems described herein can be adapted to identify the interactions in reverse, namely the ubiquitin ligase responsible for a known substrate ubiquitination. Furthermore, given that E3-substrate interactions are considered to be quite transient, the catalytic tagging strategy described herein offers an approach for identification of weaker interactions that are very difficult to detect by typical pull-down assays. The illustrated catalytic tagging system is a powerful tool for positive enrichment of substrates for IAPs. The catalytic tagging system will be generally useful for many more E3 ubiquitin ligase family members now numbering >600. More broadly, this tagging machine may be useful to identify many other transient protein complexes by linking other molecular baits to the catalytic tagging system and identifying binding partners.

### Example 2

### Screening for XIAP and cIAP1 Substrates

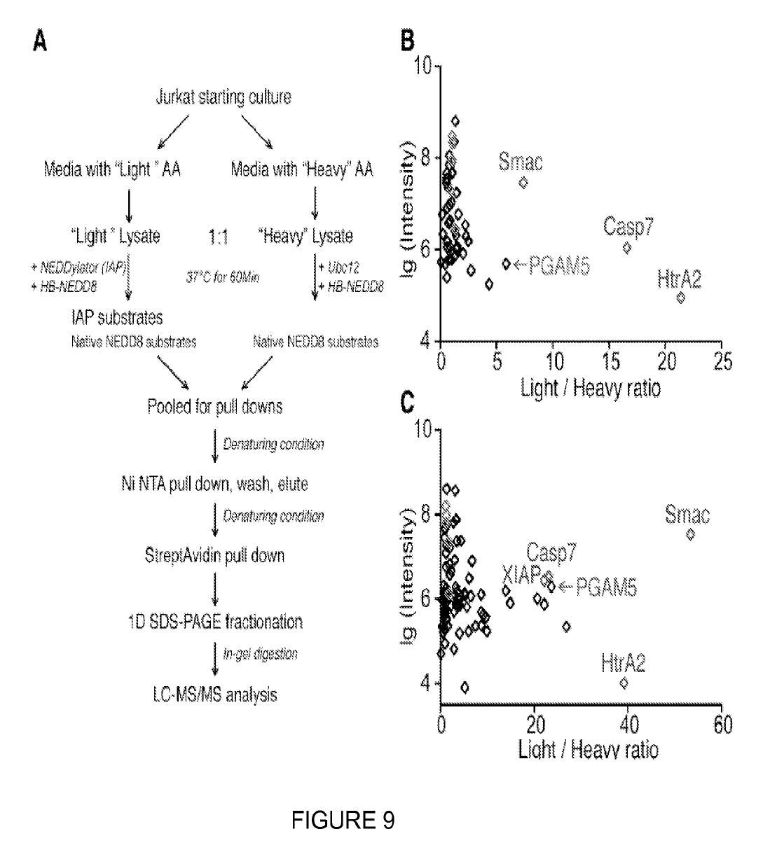

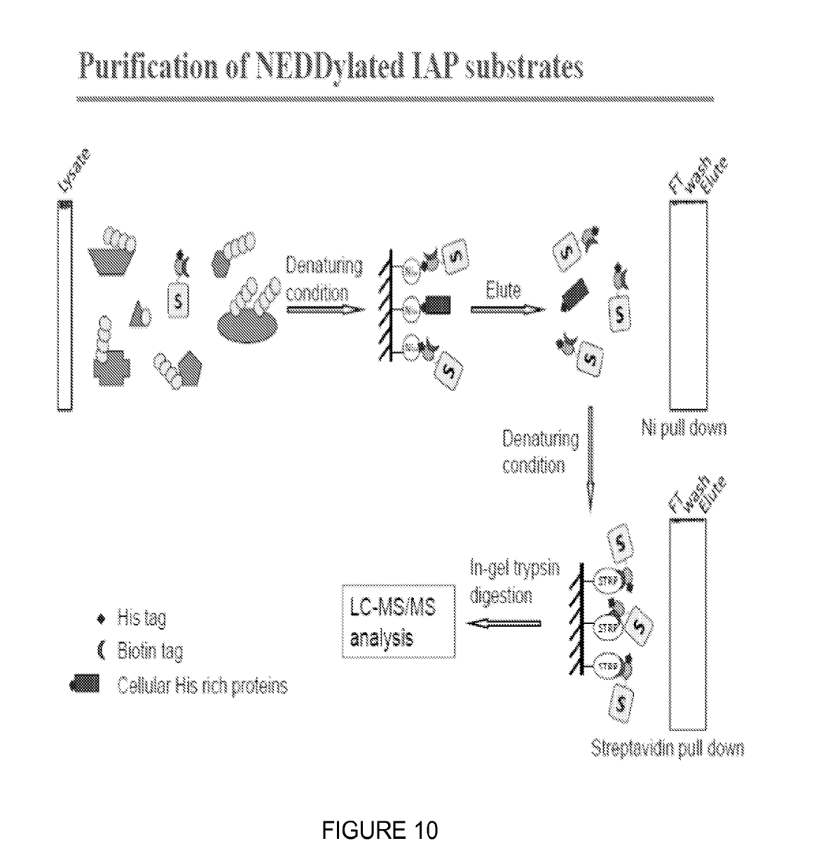

To globally identify the substrates of XIAP and cIAP1 using their respective catalytic tagging systems, stable isotope labeling by amino acid in cell culture (SILAC) (Ong and Mann, Nat Protoc 1:2650-2660 (2006))—based mass spectrometry strategy outlined in FIG. 9A was applied.

### Stable Isotope Labeling by Amino Acid in Cell Culture

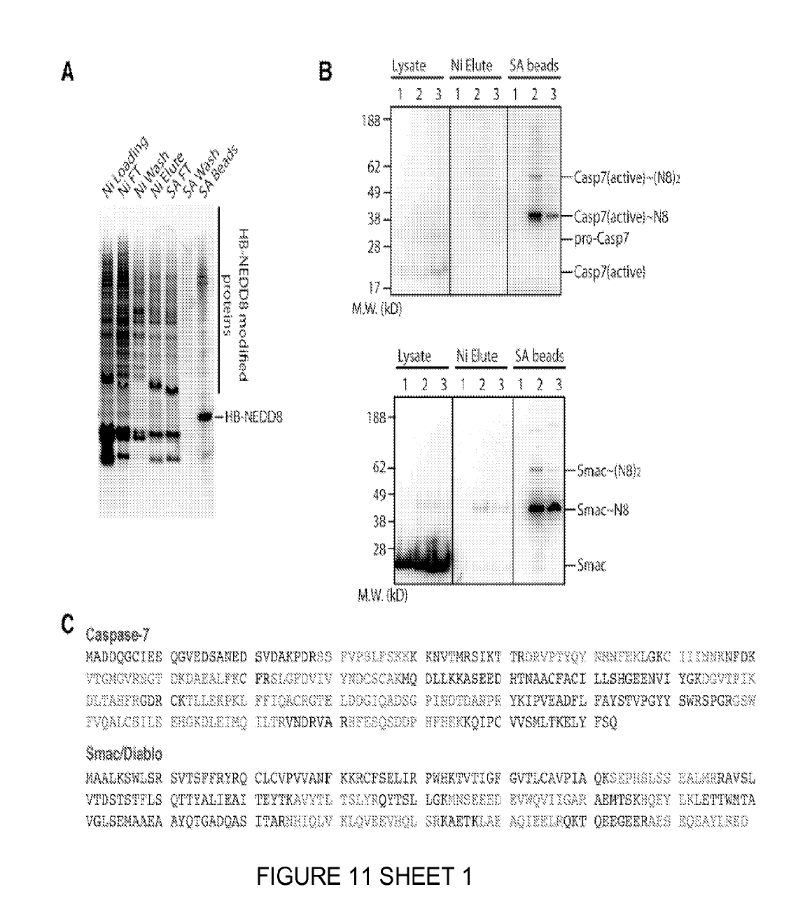

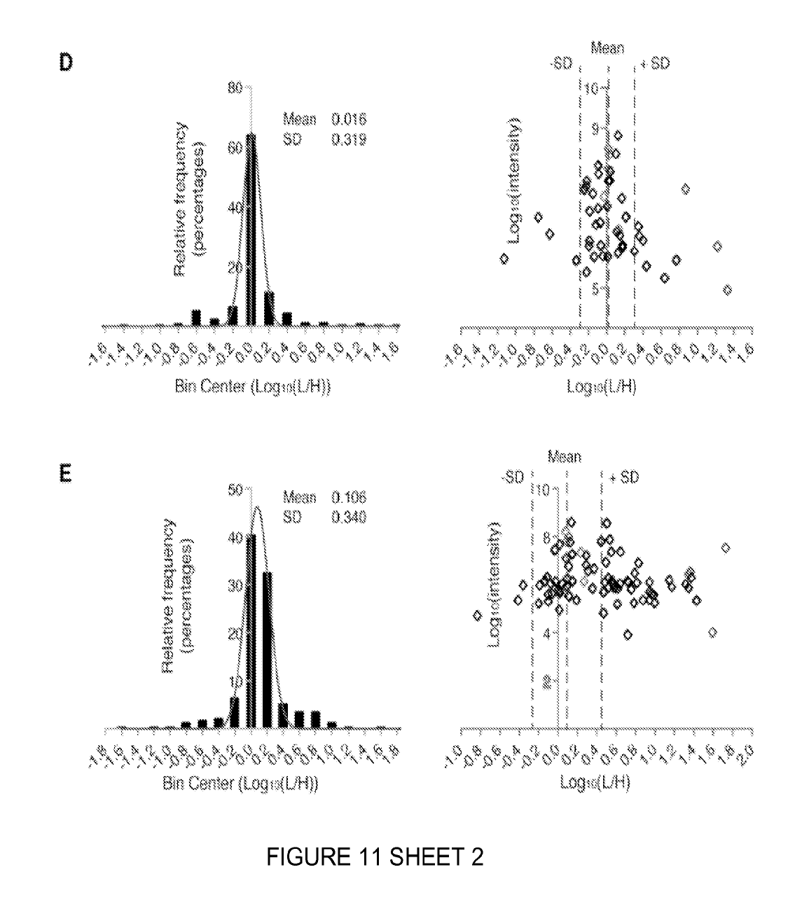

Jurkat cells were grown in media containing either light or heavy isotopes of lysine and arginine and induced to undergo apoptosis with STS. Light cell extracts were incubated with the respective catalytic tagging system for each of the two IAPs and the heavy extracts labeled only with the wild-type NEDD8 E2, Ubc12. 64 mg regular L-Lys and 83.6 mg L-Arg.HCl were added to 1 liter RPMI-1640 minus L-Lys and L-Arg (Thermo/Pierce), 10% fetal bovine serum (FBS) to generate “Light” medium. 100 mg ¹³C₆¹⁵N₂ L-Lys.2HCl and 86 mg ¹³C₆ L-Arg.HCl (Cambridge Isotope) were added to the same amount of medium to generate the “Heavy” medium. Jurkat cells were cultured separately in “Light” or “Heavy” medium for 7 days to allow for thorough isotope incorporation. For IAP substrate identification, 400 million cells were treated with 2 μM STS for 4 hours, harvested, lysed in M-PER mammalian protein extraction buffer (Thermo/Pierce) in the presence of protease inhibitors (100× ProteoBlock cocktail, Fermentas) and mixed with cell lysate prepared from 400 million untreated cells. The NEDDylated proteins were isolated under denaturing conditions via the HB-NEDD8 tag using a tandem affinity pull down procedure (Tagwerker et al., Mol Cell Proteomics 5:737-748 (2006)) (FIG. 11). Purified NEDDylated proteins were trypsinized and analyzed with tandem mass spectrometry (LC-MS/MS).

### Sample Preparation for LC-MS/MS Analysis

For SILAC experiments, Jurkat cells were cultured separately in “Light” (supplemented with regular Lys and Arg) or “Heavy” (supplemented with ¹³C₆¹⁵N₂ Lys and ¹³C₆ Arg) medium for 7 days to allow for thorough isotope incorporation. Apoptotic “Light” and “Heavy” cell extracts were prepared individually. The lysates were cleared by spinning and then diluted in 50 mM HEPES pH 7.4, 150 mM NaCl to 5 mg/ml and supplemented with 5 mM ATP, 10 mM MgCl2. Endogenous NEDD8 E1 is cell lysate was used to catalyze the reaction. HB-NEDD8 was added to both lysate at mass ratio 1:500; the catalytic tagging system was added to the “Light” lysate at mass ratio 1:1000. Equal molar of Ubc12 was added to the “Heavy” lysate. The reactions proceeded at 37° C. for 1 hour, then stopped by addition of iodoacetamide at 20 mM and solid urea to a final concentration of 8 M. “Light” and “Heavy” lysates were pooled and subjected for tandem affinity purification as described (Tagwerker et al., Mol Cell Proteomics 5:737-748 (2006)). NEDDylated proteins were separated by one-dimensional SDS-PAGE, and trypsinized for LC-MS/MS analysis.

### Mass Spectrometry and Data Analysis

For liquid chromatography—Tandem mass spectrometry (LC-MS/MS) analysis, the tryptic peptides were first desalted and concentrated with a C₁₈ Ziptip (Millipore). LC-MS/MS was carried out by reverse phase LC interfaced with a LTQ-Orbitrap (ThermoFisher Scientific) hybrid mass spectrometer. A nanoflow HPLC (NanoAcquity UPLC system, Waters Corporation) was equipped with a trap column (180 μm×20 mm, 5 μm SymmetryC₁₈, from Waters) and an analytical column (100 μm×100 mm, 1.7 μm BEH130C₁₈, from Waters). Peptides were eluted over a linear gradient from 2% solvent A (0.1% formic acid in water) to 35% solvent B (0.1% formic acid in acetonitrile) at 350 nL/min over 35 min. MS and MS/MS spectra were acquired in a data-dependent mode with MS spectra acquired in the Orbitrap at 30,000 resolving power and 6 CID MS/MS spectra acquired per MS in the LTQ linear ion trap. Dynamic exclusion was set at 90 seconds to prevent repeated MS/MS on the same precursor ion.

For data analysis, peptide sequences were assigned using the ProteinProspector database search engine against the Swiss-Prot human protein database (2011.7.6). Search parameters included a precursor mass tolerance of 30 ppm, fragment ion mass tolerance of 0.8 Da, up to 2 missed trypsin cleavages, constant carbamidomethylation of Cys, variable modifications of acetylation of protein N terminus, oxidation of Met, presence of L-Lys (¹³C₆¹⁵N₂) and L-Arg (¹³C₆) (only for SILAC experiments) and modification of Lys residues by NEDD8 (Gly-Gly, +114 m/z). The identified peptides were searched against a random decoy protein database for evaluating the false positive rates. At the maximum expectation value of peptide set at 0.1, the number of decoy peptides identified for each dataset counted for 0.73%, 1.3%, 0.2% and 0.3% respectively for XIAP_SILAC, cIAP1_SILAC, XIAP_label-free and cIAP1_labe-free experiments. The decoy peptides were removed from the data. Protein IDs were assigned only when at least two unique peptides were identified from the same protein. For SILAC experiment, Light/Heavy ratios for the identified peptides were calculated, using ProteinProspector. The media Light/Heavy ratios of all the peptides identified for each protein were used to represent the Light/Heavy ratio of the protein.

### Results

Endogenous NEDD8 substrates, and not substrates of either IAP, are expected to have Light/Heavy (L/H) ratios close to 1. In contrast the IAP substrates labeled by the respective catalytic tagging system should have high L/H ratios. In both experiments, the five cullins that are well-known NEDD8 substrates were identified with low L/H ratios ranging from 0.9 to 1.8. In stark contrast, three known IAP substrates, including caspase-7, Smac and HtrA2, had L/H ratios that were greater than 7 (FIG. 9B, C; Table 1).

TABLE 1
Potential XIAP and cIAP1 substrates identified in SILAC
experiment with Light/Heavy ratios greater than four.

| | | |
|---|---|---|
| ACC# | Protein Name | L/H |

XIAP

| | | |
|---|---|---|
| O43464 | Serine protease HtrA2, mitochondrial | 21.4 |
| P55210 | Caspase-7 | 16.6 |
| Q9NR28 | SMAC/Diablo homolog, mitochondrial | 7.39 |
| Q96HS1 | Serine/threonine-protein phosphatase PGAM5, | 5.88 |
| | mitochondrial | |
| O75934 | Pre-mRNA-splicing factor SPF27 | 4.35 |

cIAP1

| | | |
|---|---|---|
| Q9NR28 | SMAC/Diablo homolog, mitochondrial | 53.4 |
| O43464 | Serine protease HtrA2, mitochondrial | 39.2 |
| Q13148 | TAR DNA-binding protein 43 | 26.9 |
| Q96HS1 | Serine/threonine-protein phosphatase PGAM5, | 23.7 |
| | mitochondrial | |
| P55210 | Caspase-7 | 23.1 |
| P98170 | Baculoviral IAP repeat-containing protein 4 | 22.2 |
| Q9UMS4 | Pre-mRNA-processing factor 19 | 22.2 |
| Q13151 | Heterogeneous nuclear ribonucleoprotein A0 | 20.7 |
| Q92665 | 28S ribosomal protein S31, mitochondrial | 14.9 |
| O43837 | Isocitrate dehydrogenase [NAD] subunit beta, | 14 |
| | mitochondrial | |
| P82933 | 28S ribosomal protein S9, mitochondrial | 9.93 |
| P63173 | 60S ribosomal protein L38 | 9.67 |
| Q9H2W6 | 39S ribosomal protein L46, mitochondrial | 9.23 |
| Q15717 | ELAV-like protein 1 | 8.78 |
| P52566 | Rho GDP-dissociation inhibitor 2 | 8.76 |
| A6NMY6 | Putative annexin A2-like protein | 8.75 |
| Q15233 | Non-POU domain-containing octamer-binding protein | 7.52 |
| P49411 | Elongation factor Tu, mitochondrial | 6.77 |
| P48735 | Isocitrate dehydrogenase [NADP], mitochondrial | 6.51 |
| P11310 | Medium-chain specific acyl-CoA dehydrogenase, | 6.17 |
| | mitochondrial | |
| P62701 | 40S ribosomal protein S4, X isoform | 6.06 |
| P24752 | Acetyl-CoA acetyltransferase, mitochondrial | 5.67 |
| P60842 | Eukaryotic initiation factor 4A-I | 5.31 |
| P69905 | Hemoglobin subunit alpha | 5.22 |
| P04406 | Glyceraldehyde-3-phosphate dehydrogenase | 5.21 |
| P68104 | Elongation factor 1-alpha 1 | 4.4 |
| P40926 | Malate dehydrogenase, mitochondrial | 4.35 |
| Q00839 | Heterogeneous nuclear ribonucleoprotein U | 4.15 |
| Q99714 | 3-hydroxyacyl-CoA dehydrogenase type-2 | 4.1 |

To evaluate the quality of the SILAC data, the L/H ratio standard deviation (SD) was calculated from all the cullin peptides identified in each dataset (FIG. 9D, E). The observed SD was <0.35 for both datasets, which give an upper L/H ratio cutoff of 2.2 and 2.8, respectively, for the XIAP and cIAP1 datasets. Here a more stringent L/H ratio cutoff of 4 as the criteria for candidate substrates was used.

With the NEDDylator^{XIAP}, 52 proteins were identified, each having two or more unique tryptic peptides (FIG. 9B); 39 of these proteins had L/H ratios <2, probably representing the background from native NEDD8 modification. Five proteins had high L/H ratios >4, among which were the three known XIAP ubiquitin ligase substrates, caspase-7, Smac and HtrA2. With the NEDDylator^{cIAP1}, 87 proteins were identified, each having two or more tryptic peptides (FIG. 9C); 37 of which have background-level L/H ratios <2, while 29 proteins had L/H ratios >4, indicating a larger group of potential substrates for cIAP1.

The combination of the catalytic tagging system with tandem affinity pull down under denaturing conditions has very high specificity. Thus, it was tested whether substrates could be identified in unlabeled lysates without the use of SILAC methods. Indeed, peptides from the three known IAP substrates, caspase-7, Smac and HtrA2, were easily detected and only in the NEDDylator^{XIAP} or NEDDylator^{cIAP1} treated samples but not in the Ubc12 treated control samples. From this experiment, 17 additional potential substrates for XIAP and 20 for cIAP1 with ≧2 peptides were identified from each protein in the NEDDylator treated sample but not in the control samples (Table 2). Combining the non-labeled and SILAC data sets produced 18 candidate substrates for XIAP and 45 for cIAP1. XIAP and cIAP1 have 11 substrates in common (Table 1 and Table 2), consistent with their partially redundant roles in controlling apoptosis (Harlin et al., 2001). The larger number of cIAP1 substrates likely reflects its broader function in NF-κB and other signaling pathways.

TABLE 2
Potential XIAP and cIAP1 substrates identified in label-free experiement.

| | | | |
|---|---|---|---|
| | | Num | % |
| Acc # | Protein Name | Unique | Cov |

XIAP

| | | | |
|---|---|---|---|
| P55210 | Caspase-7 | 7 | 23.1 |
| P42704 | Leucine-rich PPR motif-containing protein, mitochondrial | 6 | 4.8 |
| Q9NR28 | SMAC/Diablo homolog, mitochondrial | 6 | 23.4 |
| P28482 | Mitogen-activated protein kinase 1 | 4 | 13.9 |
| P24752 | Acetyl-CoA acetyltransferase, mitochondrial | 4 | 13.3 |
| O75934 | Pre-mRNA-splicing factor SPF27 | 3 | 11.6 |
| P10809 | 60 kDa heat shock protein, mitochondrial | 2 | 3.7 |
| Q99459 | Cell division cycle 5-like protein | 2 | 3.2 |
| Q96HS1 | Serine/threonine-protein phosphatase PGAM5, mitochondrial | 2 | 7.6 |
| Q96RQ3 | Methylcrotonoyl-CoA carboxylase subunit alpha, mitochondrial | 2 | 3.9 |
| Q9NY33 | Dipeptidyl peptidase 3 | 2 | 3.8 |
| P13489 | Ribonuclease inhibitor | 2 | 5.9 |
| B3KXI2 | cDNA FLJ45429 fis, clone BRHIP3039057, highly similar to | 2 | 3.6 |
| | Protein transport protein Sec23A | | |
| Q8IWV7 | E3 ubiquitin-protein ligase UBR1 | 2 | 1 |
| Q4G0F5 | Vacuolar protein sorting-associated protein 26B | 2 | 6.5 |
| Q9H967 | WD repeat-containing protein 76 | 2 | 3 |
| O60885 | Bromodomain-containing protein 4 | 2 | 1.5 |

cIAP1

| | | | |
|---|---|---|---|
| P42704 | Leucine-rich PPR motif-containing protein, mitochondrial | 7 | 6 |
| Q99459 | Cell division cycle 5-like protein | 7 | 11.1 |
| P10809 | 60 kDa heat shock protein, mitochondrial | 5 | 10.5 |
| O75934 | Pre-mRNA-splicing factor SPF27 | 5 | 19.6 |
| Q9NR28 | SMAC/Diablo homolog, mitochondrial | 4 | 14.6 |
| Q96HS1 | Serine/threonine-protein phosphatase PGAM5, mitochondrial | 4 | 15.2 |
| Q86YZ3 | Hornerin | 3 | 2 |
| P61978 | Heterogeneous nuclear ribonucleoprotein K | 3 | 7.8 |
| Q9UQ80 | Proliferation-associated protein 2G4 | 3 | 7.9 |
| P05165 | Propionyl-CoA carboxylase alpha chain, mitochondrial | 3 | 4.1 |
| P55084 | Trifunctional enzyme subunit beta, mitochondrial | 3 | 7.2 |
| P98170 | Baculoviral IAP repeat-containing protein 4 | 2 | 9.9 |
| P02765 | Alpha-2-HS-glycoprotein | 2 | 3.5 |
| Q96RQ3 | Methylcrotonoyl-CoA carboxylase subunit alpha, mitochondrial | 2 | 4.3 |
| P24752 | Acetyl-CoA acetyltransferase, mitochondrial | 2 | 5.9 |
| P11216 | Glycogen phosphorylase, brain form | 2 | 3.7 |
| P38646 | Stress-70 protein, mitochondrial | 2 | 3.1 |
| B3KXI2 | cDNA FLJ45429 fis, clone BRHIP3039057, highly similar to | 2 | 4.1 |
| | Protein transport protein Sec23A | | |
| O14497 | AT-rich interactive domain-containing protein 1A | 2 | 1.2 |
| P27797 | Calreticulin | 2 | 5.3 |

### Example 3

### Identification of a Cleaved Form of PGAM5 with a Neo-IBM Motif Generated by a Non-Caspase Protease

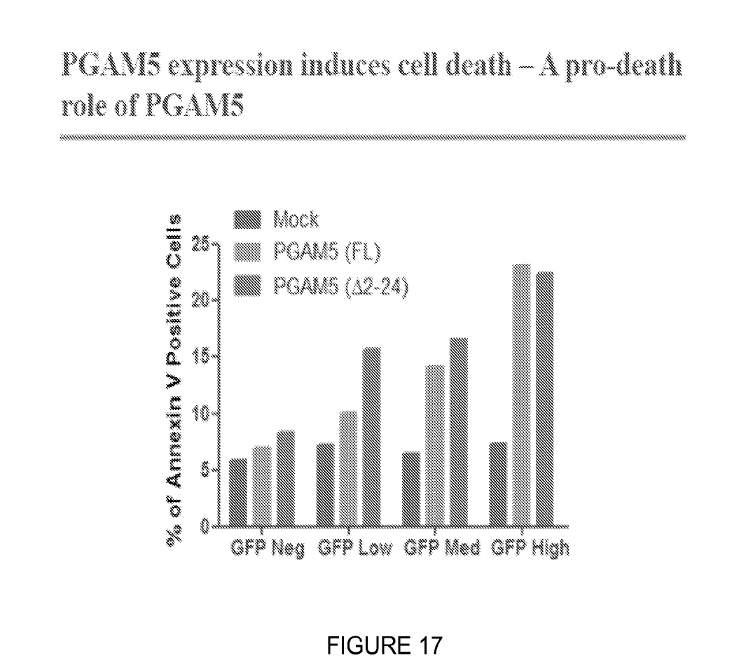

Among the proteins most strongly labeled by the IAP NEDDylators was PGAM5 (phosphoglycerate mutase family member 5), a recently discovered dimeric protein phosphatase (Takeda et al., PNAS 106:12301-12305 (2009)). PGAM5 is displayed from the outer membrane of the mitochondria through an N-terminal mitochondrial membrane peptide (Lo and Hannink, Exp Cell Res 314:1789-1803 (2008)). It is also believed to play roles in mitochondrial fission and fusion (Imai et al., PLoS Genet. 6:e1001229 (2010)) and in necrotic cell death (FIG. 17) through interaction with the RIP1/RIP3 kinases (Wang et al., Cell 148:228-43 (2012)).

There are two transcriptional isoforms of PGAM5, PGAM5L (long form) which is the most abundant and PGAM5S (short form) which lacks ˜80 amino acids at the C terminus (Lo and Hannink, 2008). Most known IAP substrates have two properties: each is processed to an active form through specific proteolysis, and proteolysis generates a new N-terminus that typically contains a tetra-peptide IBM motif (A-(V/T/I)-(P/A)-(F/Y/I/V)) (Shi, 2002). The IBM, with its invariant amino-terminal alanine, contributes to most of the binding interactions between the IAP and its substrate (Shi, Cell Death Differ 9:93-95 (2002)); Verhagen et al., Genome Biol 2 Reviews 3009 (2001)).

### Cloning and Expression of PGAM5

Full length and truncated PGAM5 (long isoform) were cloned into pET23-b vector (Novagen) between restriction sites Nde1 and Xho1 to have a C-terminal His6 tag fused in frame. Proteins were expressed in BL21 pLysS cells and purified through Ni-NTA superflow resin (Qiagen) and then buffer exchanged into 25 mM Tris pH8.0, 150 mM NaCl, 1 mM DTT with PD-10 desalting column (GE Healthcare).

### Cell Viability Assays

Jurkat cells were transfected with PGAM5 and GFP constructs, and cultured for 24 hours. Cells were then washed with phosphate-buffered saline and incubated with fluorescein-labeled annexin V reagent (annexin-V-Staining Kit, BD) for 10 min at room temperature. The percentage of annexin V positive cells was measured by flow cytometry and the data were analyzed by FlowJo softeware.

HeLa cells were plated in 96 well plates and transfected with either empty vector or the PGAM5 construct. 24 hours after transfection, cells were treated with STS for 30 min to 2 hours. CellTiter Glo (Promega) reagent was added directly to cells and incubated at room temperature for 10 min with vigorous shaking. Luminescent signals were read using a SpectraMax (Molecular Devices) plate reader.

### Results

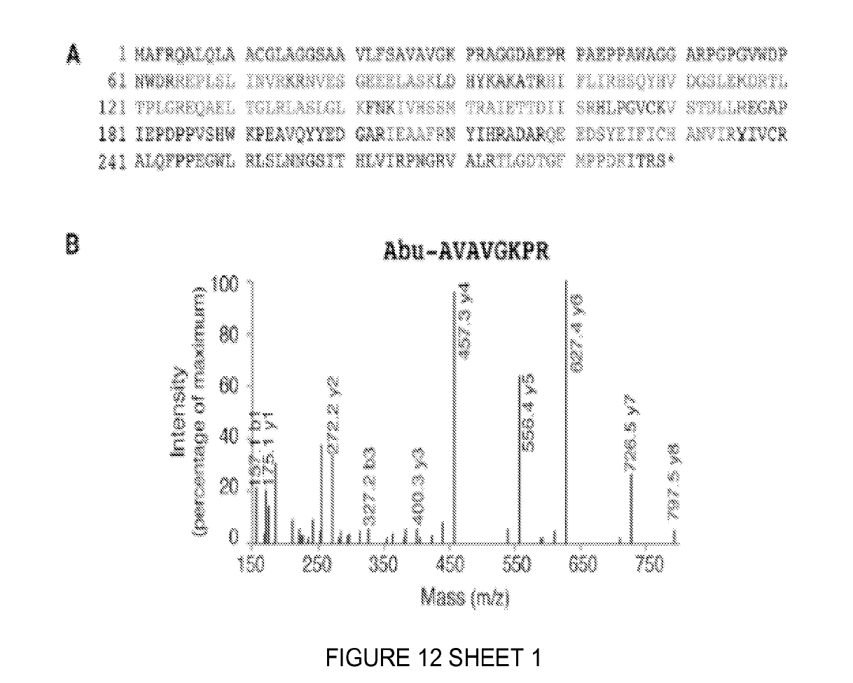

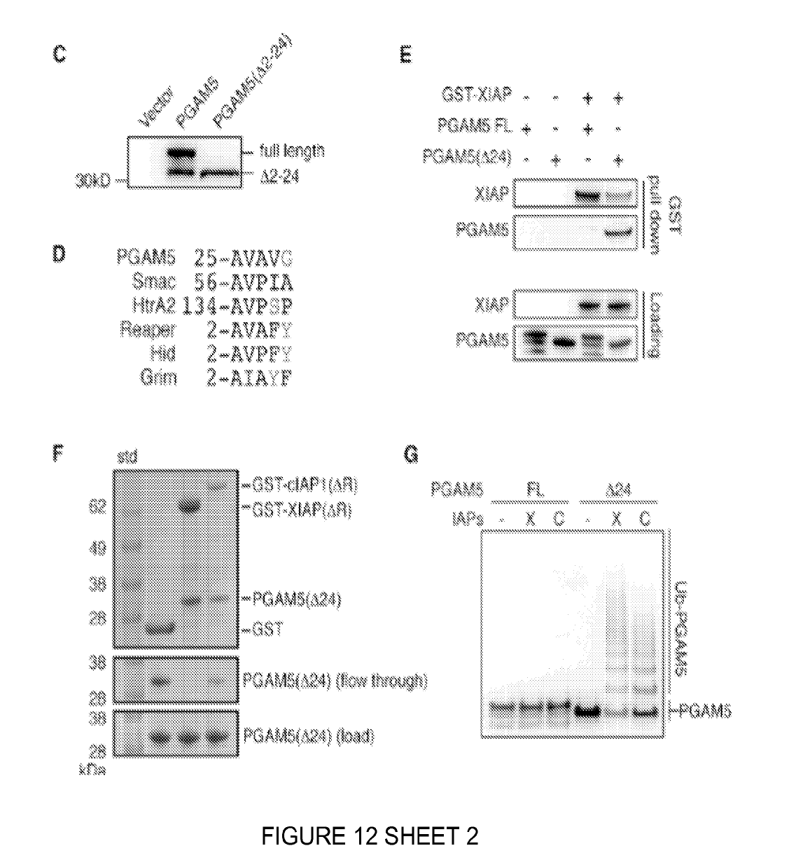

PGAM5 was found as a potential substrate for both XIAP and cIAP1 in four independent biological experiments with strong coverage (FIG. 12A). It was also identified in SILAC based experiments with very high L/H ratios like those of known IAP substrates (FIG. 9B, C). The C terminal unique peptides (residues 274˜285) were identified from PGAM5L (FIG. 12A), showing the presence of PGAM5L. Proteomics experiments cannot otherwise distinguish these forms and they are referred to collectively as PGAM5.

To test if the neo-IBM motif is required for PGAM5 to interact with IAPs, it was determined which form of PGAM5 is the substrate of IAP. To express and purify PGAM5(Δ24) in bacteria with the neo-IBM N-terminal sequence, PGAM5(Δ2-24) was cloned with N-terminal sequence of Met-Ala-Val-Ala-Val and C-terminal His tag.

A cleaved form of PGAM5 lacking the first 24 residues was identified in four different cell lines undergoing apoptosis (FIG. 12B, Table 3) using the subtiligase-based N-terminal labeling technique (Mahrus et al., Cell 134:866-876 (2008); Shimbo et al., PNAS 109:12432-12437 (2012)). The cleavage does not derive from a caspase because the P1 residue is serine, not aspartate. The presence of this truncated PGAM5 was validated in cells by immuno-blotting (FIG. 12C). Substrate validation experiments were all performed with the long isoform. The truncated PGAM5 identified contains a putative neo-IBM sequence starting with residue Ala25 (FIG. 12D).

TABLE 3
PGAM5 peptide identified in different
N-terminomics experiments from four different cell
lines and treated with different perturbants

| Cell type | Perturbation | Peptide |
|---|---|---|
| Jurkat | — | SerTyr-AVAVGKPR |
| Jurkat | Doxorubicin | SerTyr + iTRAQ-AVAVGK |
| Jurkat | Staurosporine | Abu-AVAVGKPR |
| THP-1 | — | SerTyr-AVAVGKPR |
| THP-1 | Caspase-1 | SerTyr-AVAVGKPR |
| THP-1 | MSU | SerTyr-AVAVGKPR |
| THP-1 | LPS + ATP | SerTyr-AVAVGKPR |
| MM1-S | bortezomib | Abu-AVAVGKPR |
| DB | 1541B | Abu-AVAVGKPR |

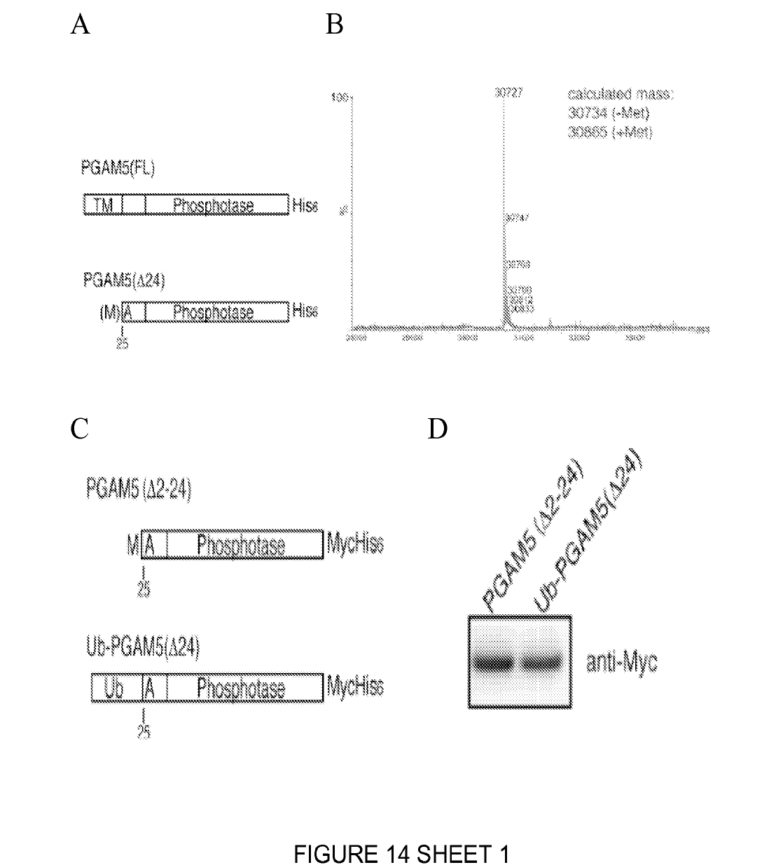

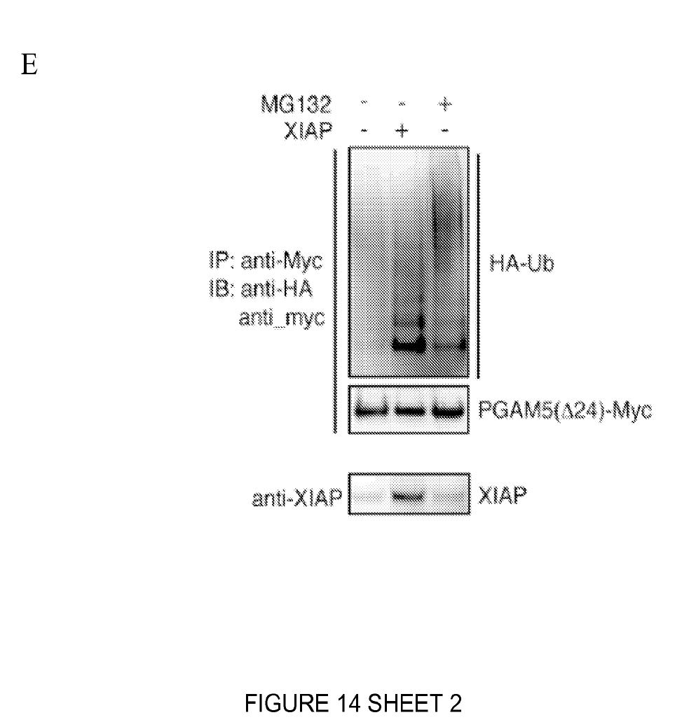

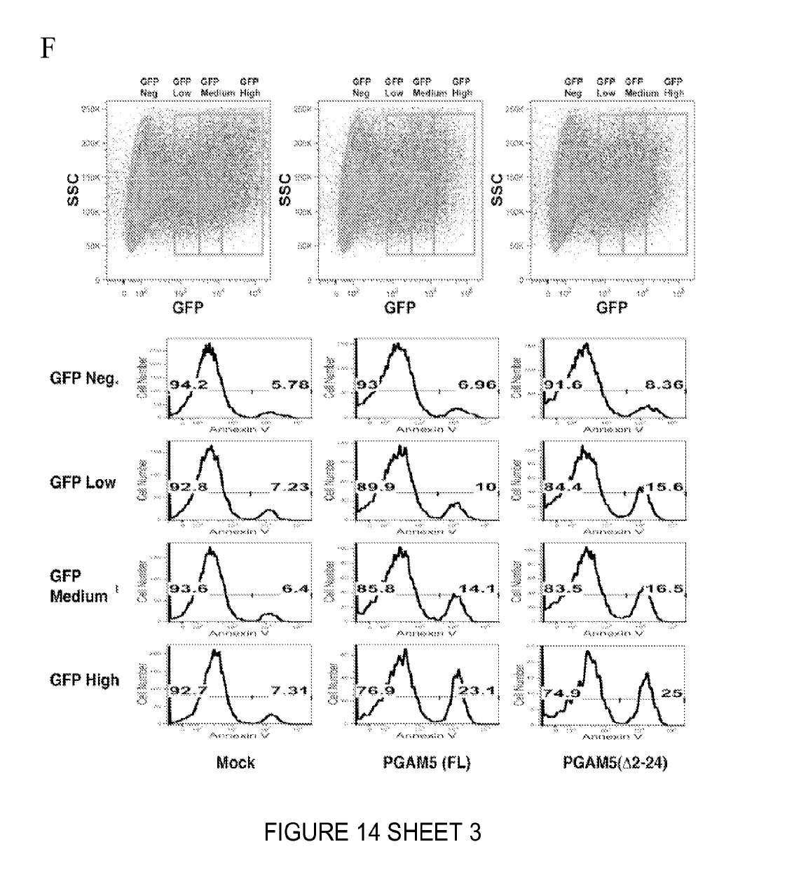

During protein expression in E. coli the first Met is found to be fully cleaved, presumably by the endogenous methionyl aminopeptidase (MAP) (Hirel et al., PNAS 86:8247-8251 (1989)), to generate the authentic PGAM5(Δ24) (FIG. 14A). In the in vitro binding experiments, PGAM5(Δ24) and not the full length PGAM5 was pulled down by a GST-XIAP fusion protein (FIG. 12E). The interaction between IAPs and PGAM5(Δ24) is dependent on the neo-IBM motif and not the RING domain (FIG. 12F). Furthermore, XIAP and cIAP1 ubiquitinates the truncated PGAM5(Δ24) and not the full length protein in the in vitro ubiquitination assay (FIG. 12G). PGAM5 ubiquitination (FIG. 12G) was assayed in the presence of 50 nM UBE1, 200 nM UbcH5b, 700 nM XIAP or cIAP1, and 1.5 μM PGAM5-His6.

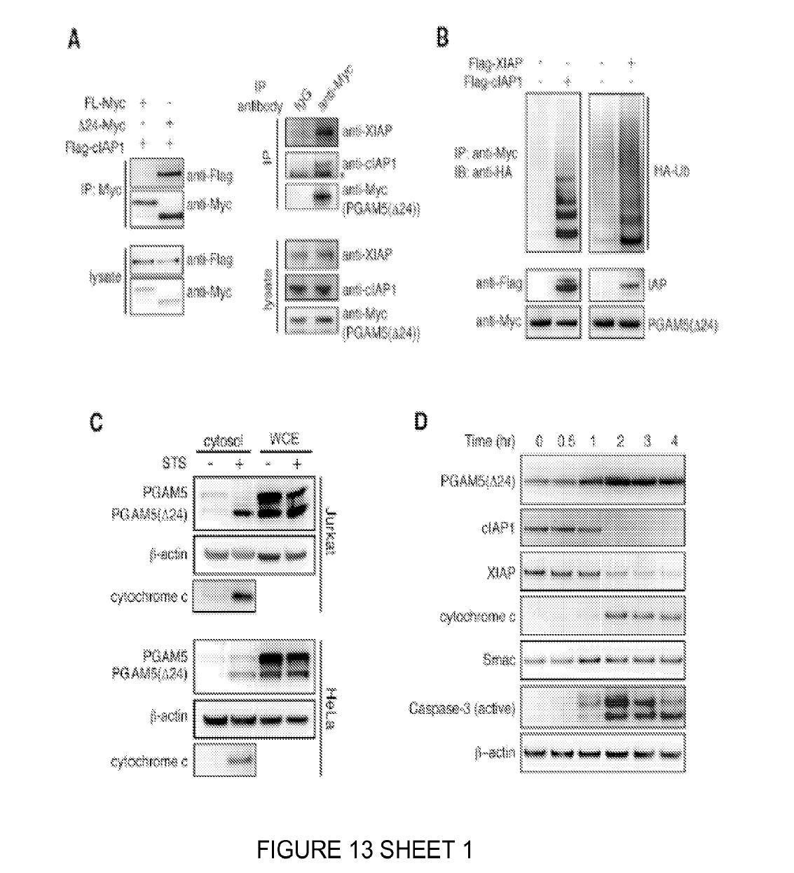

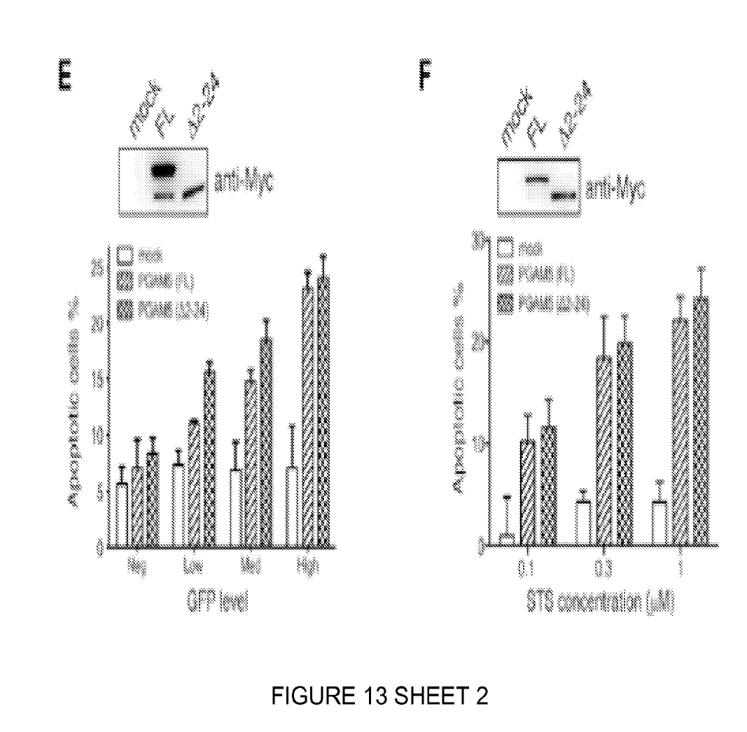

It was next addressed whether IAPs can bind and ubiquitinate PGAM5(Δ24) in cells. An N-terminal Ala is an invariant determinant in all known IBM motifs. Thus it was necessary to express PGAM5(Δ24) with its proper neo-IBM motif exposed in mammalian cells without commensurate N-terminal initiator methionine or subsequent acetylation typical seen in cytosolic proteins. To achieve this, the N-terminal ubiquitin fusion technique was used, where ubiquitin is co-translationally cleaved at the last Gly residue to expose the desired N-terminal sequence of the fused protein (Varshaysky, Genes Cells 2:13-28 (1997)). This technique has been used to directly express the Smac containing exposed IBM motif with its transit peptide removed (Hunter et al., J. Biol. Chem. 278:7494-7499 (2003)). Immuno-blotting confirmed the complete cleavage of the N-terminal ubiquitin and generation of the truncated form of PGAM5(Δ24) in cells (FIG. 14B). Consistent with the results from in vitro binding experiments (FIG. 12E), cellular cIAP1 specifically binds PGAM5(Δ24) but not the full length protein (FIG. 13A, left panels). Moreover, endogenous XIAP and cIAP1 were found to co-immunoprecipitate with PGAM5(Δ24) (FIG. 13A, right panels). Ubiquitination of PGAM5(Δ24) was stimulated by over-expression of XIAP or cIAP1 (FIG. 13B). In addition, the level of ubiquitination of PGAM5(Δ24) increased in the presence of proteasome inhibitor MG132 indicating the proteasome degrades the ubiquitinated product of PGAM5(Δ24) (FIG. 12C).

It was next determined whether PGAM5 is a Neo-IBM class mitochondrial IAP substrate. Classic substrates like Smac and HtrA2 are imported into the mitochondrial inter-membrane space where the N-terminal signal sequence is removed to produce the mature forms with IBM motifs. They reside sequestered from the cytosolic IAPs until they are released through changes in mitochondrial outer membrane permeability during apoptosis. In stark contrast the N terminus of full-length PGAM5 masks the internal IBM motif at residue 25 and anchors it to the outer membrane of the mitochondria. In the non-apoptotic cytosol, there is a low level of PGAM5 that is comparable to Smac. However, upon induction of apoptosis with STS there is a significant increase in PGAM5(Δ24) in the cytosolic level that resembles the kinetics for Smac and cytochrome c release (FIG. 13C, D). Moreover, the release of PGAM5 proceeds the generation of active caspase-3 and subsequent loss of XIAP and cIAP1 as apoptosis proceeds (FIG. 13D). These data suggest PGAM5 release from the mitochondrial is an early event in apoptosis coincident with release of cytochrome c and Smac. In healthy cells by contrast there are significant amounts of cleaved PGAM5 in whole cell extracts but little in the cytosolic fraction (FIG. 13C). This suggests a portion of the cleaved PGAM5 is still sequestered on mitochondria. It is not clear whether the large increase of cytosolic PGAM5(Δ24) during apoptosis is due to more proteolysis or from translocation of a pre-cleaved PGAM5(Δ24) from the mitochondria.

Expression of Smac is known to sensitize cells to apoptosis stimuli (Du et al., Cell 102:33-42 (2000)), and expression of HtrA2 causes atypical cell death (Suzuki et al., 2001a). It was found that high PGAM5 expression levels in cells are similarly correlated with cell death. Exogenous expression of PGAM5(Δ2-24) in cell cytosol alone can induce cell death that is as effective as expression of the full length PGAM5 (FIG. 13E, FIG. 14D). Like Smac, transient expression of PGAM5 sensitized cells to the apoptosis inducer STS. The cytosolic PGAM5(Δ2-24) is as efficient as the full length PGAM5 at inducing cell death (FIG. 13F). Overall, these data suggest an important regulatory role of the cleaved form of PGAM5 in apoptosis.

### Discussion

Without being bound by theory, several possible mechanisms can account for the pro-apoptotic function of the cleaved form of PGAM5. The most direct is that PGAM5(Δ24), which contains an IBM motif, would antagonize binding of IAPs to activated capsases and thus foment apoptosis. Second, the release of PGAM5(Δ24) to the cytosol could lead to a significant change in substrate profiles both on the mitochondria and throughout the cell. The mitochondrial PGAM5 was recently proposed as a regulator of induced necrotic cell death (Wang et al., 2012). This was studied by simultaneous addition of TNFα to activate the TNFα receptor, addition of a Smac mimetic compound to block cIAP1 and cIAP2, and by addition of z-VAD-fmk to block caspase activity. Under these conditions it was found that RIP1/RIP3 kinases, critical in driving cellular necrosis, associate with mitochondria bound PGAM5. However during apoptosis the data show that PGAM5 is released early on and could steer the cell away from necrotic cell death by preventing mitochondrial binding of RIP kinases.

### Example 4

### Mammalian Mitochondrial Proteins as Potential IAP Substrates

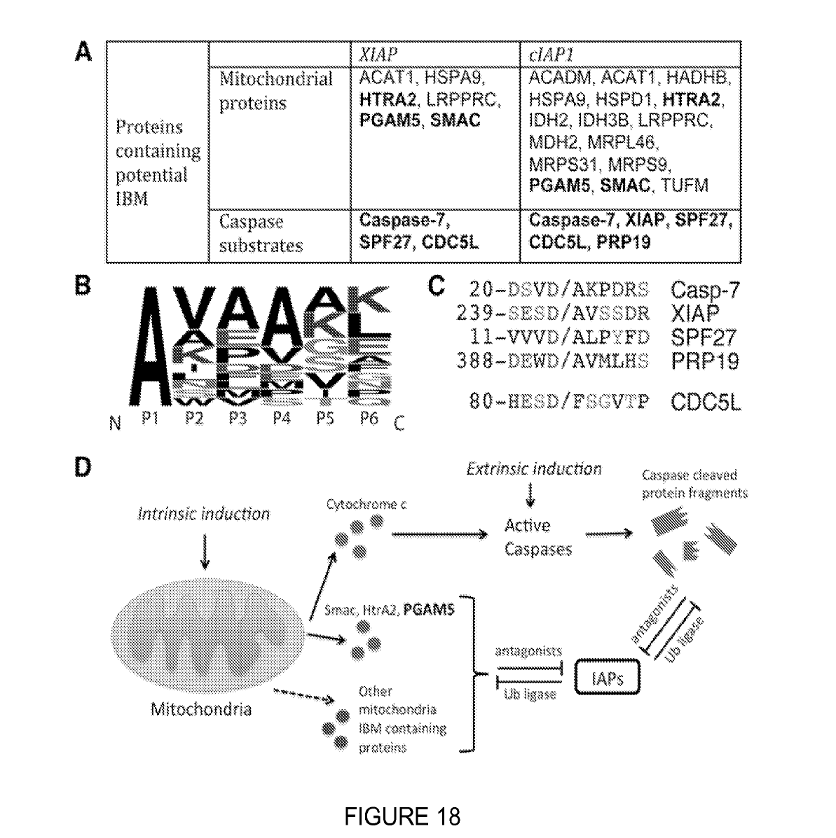

In addition to Smac, HtrA2 and PGAM5, a large portion of the newly identified XIAP and cIAP1 binding proteins are also mitochondrial proteins (FIG. 18A). Using N-terminomics technology (Mahrus et al., Cell 134, 866-876 (2008)), a large experimental degradomic database has been generated, where the free N-termini of proteins that result from apoptosis have been identified. By searching against this database, it was found that among the 16 mitochondrial proteins labeled by the NEDDylator^{cIAP1}, 15 are processed into mature forms with the N-terminal mitochondrial targeting transit peptide proteolytically removed, similar to Smac and HtrA2 (Table 4). Interestingly, all the cleaved forms of these mitochondrial proteins contain an N-terminal alanine and an IBM motif-like structure (FIG. 18B). The interaction between XIAP and one of these mitochondrial proteins, LRPPR, has been validated in cells (Verhagen et al., 2007).

TABLE 4
Mitochondrial proteins containing potential IBM motifs

| ACC # | Gene Name | Protein Name | Peptide identified in Degrabase |
|---|---|---|---|
| Q9NR28 | DIABLO | SMAC/Diablo homolog, mitochondrial | 56-AVPIAQK |
| O43464 | HtrA2 | Serine protease HtrA2, mitochondrial | 134-AVPSPPPASPR |
| Q96HS1 | PGAM5 | Serine/threonine-protein phosphatase PGAM5, | 25-AVAVGKPR |
| | | mitochondrial | |
| P42704 | LRPPRC | Leucine-rich PPR motif-containing protein, | 60-AIAAKEKDIQEESTFSSR |
| | | mitochondrial | |
| P10809 | HSPD1 | 60 kDa heat shock protein, mitochondrial | 27-AKDVKFGADAR |
| P55084 | HADHB | Trifunctional enzyme subunit beta, | 34-AAPAVQTK |
| | | mitochondrial | |
| P38646 | HSPA9 | Stress-70 protein, mitochondrial | 47-ASEAIK |
| Q92665 | MRPS31 | 28S ribosomal protein S31, mitochondrial | 161-AVADSLPFDKQTTK |
| O43837 | IDH3B | Isocitrate dehydrogenase [NAD] subunit | 301-AVFETGARHPFAQAVGR |
| | | beta, mitochondrial | |
| P82933 | MRPS9 | 28S ribosomal protein S9, mitochondrial | 89-ANMMGEDPETFTQEDIDR |
| Q9H2W6 | MRPL46 | 39S ribosomal protein L46, mitochondrial | 33-ALAAAPSSNGSPWR |
| | | | 36-AAPSSNGSPWR |
| P49411 | TUFM | Elongation factor Tu, mitochondrial | 44-AVEAKKTYVR |
| P48735 | IDH2 | Isocitrate dehydrogenase [NADP], | 20-AWAPAALTAPTSQEQPR |
| | | mitochondrial | |
| P11310 | ACADM | Medium-chain specific acyl-CoA | 27-ANRQRE (predicted) |
| | | dehydrogenase, mitochondrial | |
| P24752 | ACAT1 | Acetyl-CoA acetyltransferase, mitochondrial | 2-AVLAALLR |
| P40926 | MDH2 | Malate dehydrogenase, mitochondrial | 25-AKVAVLGASGGIGQPLSLLLK |

Caspases are the cysteine proteases that specifically cleave at carboxyl side of aspartic acid and drive the final stages of apoptosis. Global proteomics studies have shown that caspases can cleave>1000 substrates during apoptosis (Agard et al., PNAS 109:1913-1918 (2012); Dix et al., Cell 134:679-691 (2008); Mahrus et al., Cell 134:866-876 (2008); Shimbo et al., PNAS 109:12432-12437 (2012)). Five substrates of cIAP1 were identified that are known targets of caspases (FIG. 17A): SPF27 (pre-mRNA-splicing factor SPF27), PRP19 (pre-mRNA-processing factor 19), CDC5L (cell division cycle 5-like), XIAP and caspase-7. cIAP1 is known to mediate ubiquitination and degradation of XIAP, through the heterodimerization of RING domains from each protein (Silke et al., PNAS 102:16182-16187 (2005)). However, the NEDDylator^{cIAP1} lacks the RING domain yet robustly tags XIAP. It is known that caspase-mediated proteolysis of XIAP actually generates an IBM-like sequence (Deveraux et al., EMBO 18:5242-5251 (1999)). Thus, it is reasonable that this neo-IBM motif, not the ring domain, binds cIAP1 during apoptosis when caspases are active without the need for RING-domain assisted heterodimerization with cIAP1. The same binding mechanism may occur for the other two caspase substrates, SPF27 and PRP19, both of which were found cleaved in STS treated Jurkat cells and both exposed an neo-IBM motif upon caspase cleavage (FIG. 17C) (Mahrus et al., Cell 134:866-876 (2008)). Although CDC5L does not contain an IBM motif, it is known to form a complex important for mRNA splicing with IBM-containing SPF27 and PRP19 (Ajuh et al., EMBO 19:6569-6581 (2000)). Thus, these data suggest the exemplary catalytic tagging systems can also indirectly tag bound proteins in complexes, which is known to occur for other ubiquitin ligases (Hao et al., Mol Cell 20:9-19 (2005)).

Caspase-cleaved protein fragment binding to IAPs reflects a potential feed forward mechanism to antagonize IAPs and further activate caspases. Indeed, >100 proteins were found that are cleaved by caspases during apoptosis that generate a neo-N-terminal alanine (Shimbo et al., PNAS 109:12432-12437 (2012)), an important characteristic of IBM motifs. This is an important alternative mechanism for enhancing caspase-8 mediated type-I cell death that does not involve immediate release of Smac and HtrA2 from the mitochondria.

These studies increase by more than eight-times the number of reported substrates for XIAP and cIAP1 that are generated during apoptosis. Additional studies using the catalytic tagging systems described herein will reveal other inducer-specific IAP substrates both for extrinsic apoptosis, necroptosis, inflammosis and perhaps other inducer-specific signaling events mediated by IAPs.

In addition to Smac, HtrA2, and PGAM5, 13 mitochondrial proteins were identified that contain potential IBM motifs. Previous studies using traditional immuno-precipitation coupled with mass spectrometry identified five mitochondrial proteins bearing IBMs (Verhagen et al., 2007). These proteins were shown to bind XIAP including LRPPR that was found here.

### Example 4

### Catalytic Tagging Systems that Identify Targets of Small Molecule Targets

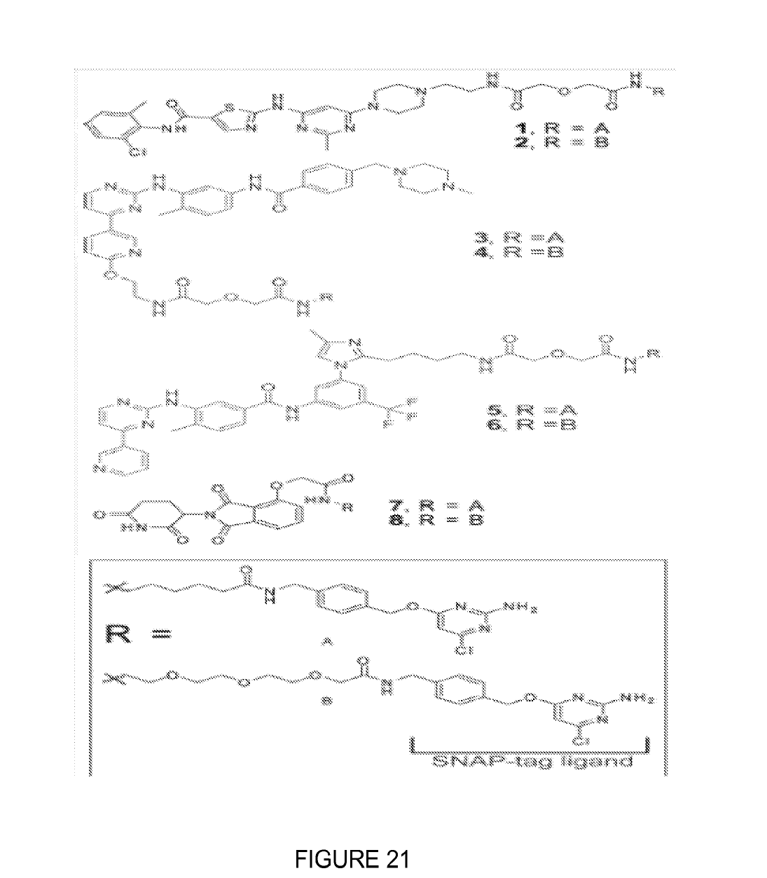

The catalytic tagging system can be used to detect protein targets of bioactive small molecules. By genetically fusing the self-labeling protein, SNAP-tag (Takeda et al., PNAS 106:12301-12305 (2009)), to Ubc12 a catalytic tagging system is generated that is capable of displaying a small molecule (NEDDylator^{SM}) (FIG. 19A). Two fusions were generated, one with a flexible 4 amino acid linker (SGSG) and one with a 10 amino acid (SGSGSGSGSG) linker (FIG. 19B). As proof of principal, the potent inhibitor of kinases dasatinib was used. Thus, dasatinib provides an ideal system for optimizing the use of the NEDDylator^{SM}. Because dasatinib can be modified at its piperazine ring with minimal effect on binding, this site was for linker attachment. To ensure that dasatinib is displayed from the SNAP-tag domain at a distance capable of binding its target proteins, Compound 1 of FIG. 21 was synthesized (FIG. 21, compound 1). A second compound (FIG. 21, compound 2) will be synthesized for linker length diversity.

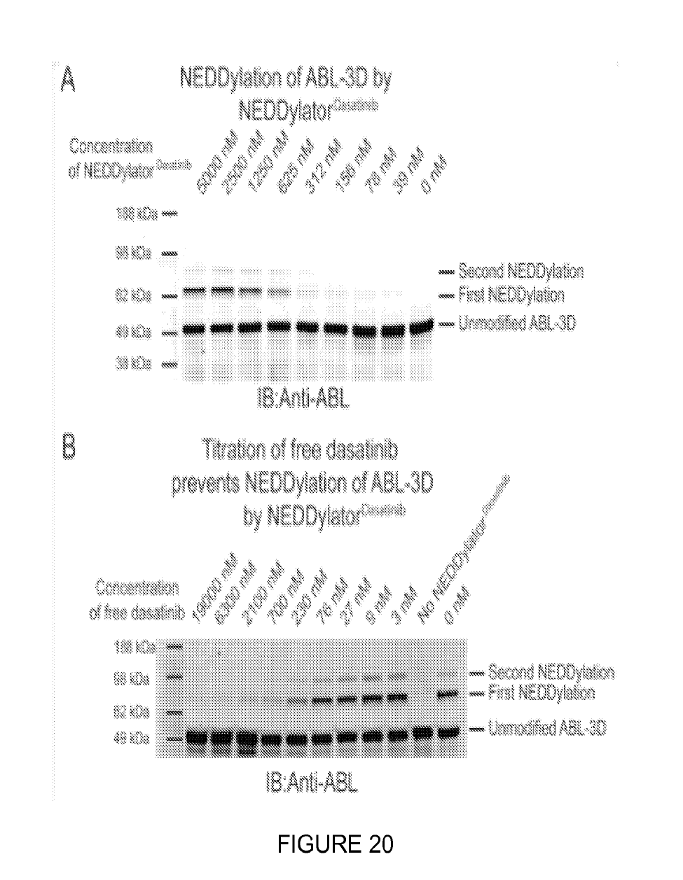

A truncated form of ABL kinase (ABL-3D, SH3-SH3-Kinsase domains) was used. In these experiments, a single NEDDylator^{Dasatinib} construct is produced by reacting with Compound 1 using standard SNAP-tag labeling conditions. Incubation for 2 hours with excess Compound 1 at room temperature led to greater than 90% conjugation as assessed by mass spectrometry (data not shown). The NEDDylator^{Dasatinib} was incubated with ATP, NEDD8, NEDD8 E1, and ABL kinase. After 2 hours, the reaction was stopped and analyzed by immunoblotting for ABL kinase. Higher weight bands of ABL kinase were observed, indicating successful NEDDylation (FIG. 20A). The extent of NEDDylation was comparable to that seen in vitro for NEDDylator^{XIAP} on its known substrate, capsase-7. Both the 10 amino acid and 4 amino acid linker construct of the NEDDylator showed similar NEDDylation of ABL (data not shown). Importantly, if the NEDDylator^{Dasatinib} construct concentration is held constant and free dasatinib is titrated into the reaction, NEDDylation is inhibited, indicating that NEDDylation of ABL by the NEDDylator^{Dasatinib} is driven by the affinity of dasatinib for ABL (FIG. 20B). SNAP-tag ligand derivatives of imatinib (FIG. 21, 3-4) and nilotinib (FIG. 21, 5-6) will also be generated to identify binding substrates to those compounds.

### Example 5

### Identification of Small Molecule Targets in Living Cells

Small molecules can also be used to identify direct protein targets of small molecules in living cells because the small molecule catalytic tagging system is genetically encoded and can be conjugated with the small molecule in cells using the SNAP-tag, although any tag that will conjugate small molecules to the catalytic tagging systems described herein can be used. This allows the identification of targets in situ and in controlled cellular states. The small molecule catalytic tagging system can be introduced in cells by transient or stable transfection. Because the compounds are cell permeable and SNAP-tag labeling is well established in cells, the labeled-small molecule catalytic tagging system can be self-assembled in living cells. SILAC experiments using cells expressing small molecule catalytic tagging system constructs as well as NEDD8 can be performed. These experiments will allow for identification of direct protein targets in the most physiologically relevant environment possible. The target profile of dasatinib, imatinib, and nilotinib in cell lysates will be compared to that in living cells while using the same cell line. Because the small molecule catalytic tagging system protein construct will stay constant and only the derivatized drug will change as the scope of this technology is expanded beyond dasatinib, imatinib, and nilotinib, a number of stable cell lines can be generated that will express the small molecule catalytic tagging system as well as NEDD8. These cell lines will have applications in identifying the targets of a wide range of small-molecule drugs including the thalidomide derivatives described above.

The examples set forth above are provided to give those of ordinary skill in the art a complete disclosure and description of how to make and use the embodiments of the compositions, systems and methods of the disclosure, and are not intended to limit the scope of what the inventors regard as their disclosure. Modifications of the above-described modes for carrying out the disclosure that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

What is claimed is:

1. An assay for the identification of a substrate comprising:
fusing a bait to an E2 ubiquitin-like conjugating enzyme wherein under wild-type conditions said E2 ubiquitin-like conjugating enzyme conjugates a ubiquitin-like protein other than ubiquitin;
expressing in a cell said bait fused to said E2 ubiquitin-like conjugating enzyme wherein the cell comprises one or more ubiquitin-like proteins; and
identifying a substrate, wherein said substrate binds ubiquitin under wild-type conditions and is identified by selecting said substrate bound to said one or more ubiquitin-like proteins.

2. The assay of claim 1, wherein the bait comprises a peptide, a protein, a small molecule, a nucleic acid, or a carbohydrate.

3. The assay of claim 1, wherein the bait is a modified version of an E3 ligase and wherein the E3 ligase lacks a RING domain.

4. The assay of claim 3, wherein the E3 ligase is selected from the group consisting of XIAP, cIAP1, CRBN, gp78, Doa10, RNF13, RNF38, TEB4, RNF139, RNFx2, RNF126, Hrd1, and MARCH1.

5. The assay of claim 4, wherein the E3 ligase is XIAP.

6. The assay of claim 4, wherein the E3 ligase is cIAP1.

7. The assay of claim 3, wherein the modified version of the E3 ligase comprises a substrate binding domain and wherein the modified version of the E3 ligase is capable of fusing to an E2 ubiquitin-like conjugating enzyme.

8. The assay of claim 1, wherein the bait is a phosphatase.

9. The assay of claim 1, wherein the bait is a kinase.

10. The assay of claim 1, wherein the bait is a small molecule.

11. The assay of claim 10, wherein the small molecule is selected from the group consisting of dasatinib, imatinib, nilotinib, thalidomide, lenalidomide, and pomalidomide.

12. The assay of claim 11, wherein the small molecule is dasatinib.

13. The assay of claim 1, wherein the ubiquitin-like protein is selected from the group consisting of NEDD8, SUMO, ISG15, ATG8, ATG12, FAT10 and functional equivalents thereof.

14. The assay of claim 13, wherein the ubiquitin-like protein is NEDD8.

15. The assay of claim 1, wherein the E2 ubiquitin-like conjugating enzyme is selected from the group consisting of NEDD8 E2, SUMO E2, ISG15 E2, ATG8 E2, ATG12 E2, FAT10 E2, and functional equivalents thereof.

16. The assay of claim 1, wherein the E2 ubiquitin-like conjugating enzyme is NEDD8 E2 or a functional equivalent thereof.

17. The assay of claim 15, wherein the NEDD8 E2 is Ubc12.

18. The assay of claim 1, wherein the substrate is specific for the bait.

19. The assay of claim 1, wherein the identifying comprises taking a cell extract and screening candidate substrates using mass spectrometry.

20. The assay of claim 12, wherein the mass spectrometry is SILAC mass spectrometry.

21. The assay of claim 12, wherein the mass spectrometry is liquid chromatography-tandem mass spectrometry (LC/MS/MS).

22. A composition comprising a bait, wherein said bait is fused to an E2 ubiquitin-like conjugating enzyme capable of conjugating a ubiquitin-like protein other than ubiquitin.

23. The composition of claim 22, wherein the bait comprises a peptide, a protein, a small molecule, a nucleic acid, or a carbohydrate.

24. The composition of claim 22, wherein the bait is a modified version of an E3 ligase and wherein the E3 ligase lacks a RING domain.

25. The composition of claim 24, wherein the E3 ligase is selected from the group consisting of XIAP, cIAP1, gp78, Doa10, RNF13, RNF38, TEB4, RNF139, RNFx2, RNF126, Hrd1, and MARCH1.

26. The composition of claim 24, wherein the E3 ligase is XIAP.

27. The composition of claim 24, wherein the E3 ligase is cIAP1.

28. The composition of claim 24, wherein the modified version of the E3 ligase comprises a substrate binding domain and wherein the modified version of the E3 ligase is capable of fusing to an E2 ubiquitin-like conjugating enzyme.

29. The composition of claim 22, wherein the bait is a phosphatase.

30. The composition of claim 22, wherein the bait is a kinase.

31. The composition of claim 22, wherein the bait is a small molecule.

32. The composition of claim 31, wherein the small molecule is selected from the group consisting of dasatinib, imatinib, nilotinib, thalidomide, lenalidomide, and pomalidomide.

33. The assay of claim 32, wherein the small molecule is dasatinib.

34. The composition of claim 22, wherein the E2 ubiquitin-like conjugating enzyme is selected from the group consisting of NEDD8 E2, SUMO E2, ISG15 E2, ATG8 E2, ATG12 E2, FAT10 E2 and functional equivalents thereof.

35. The composition of claim 34, wherein the E2 ubiquitin-like conjugating enzyme is a NEDD8 E2 or a functional equivalent thereof.

36. The composition of claim 35, wherein the NEDD8 E2 is Ubc12.

37. The composition of claim 22, wherein the ubiquitin-like protein is selected from the group consisting of NEDD8, SUMO, ISG15, ATG8, ATG12, FAT10, and functional equivalents thereof.

38. The composition of claim 37, wherein the ubiquitin-like protein is NEDD8 or a functional equivalent thereof.

39. The composition of claim 38, wherein the NEDD8 is biotinylated.

40. A kit comprising the composition of claim 22.

41. The kit of claim 40, wherein the kit further comprises a ubiquitin-like protein selected from the group consisting of NEDD8, SUMO, ISG15, ATG8, ATG12, FAT10, and a functional equivalent thereof.

42. The kit of claim 40, wherein the ubiquitin-like protein is NEDD8 or a functional equivalent thereof.

43. A method of inducing apoptosis in a cell of a subject comprising administering to the subject an effective amount of one or more substrates identified by the assay of claim 3.

44. The method of claim 43, wherein the substrate is PGAM5.
